(19) United States
(12) Patent Application Publication
(10) Pub. No.: US 2026/0351401 A1
(43) Pub. Date: Nov. 5, 2026

(54) SYSTEMS AND METHODS FOR MEDICAL SYSTEM REQUEST PROCESSING

(71) Applicants: Boston Scientific Scimed, Inc., Maple Grove, MN (US); Boston Scientific Medical Device Limited, Galway (IE)

(72) Inventors: Zhuolin WU, Circle Pines, MN (US); Veerakumar VENUGOPAL, Carver, MN (US); Michael Francis MCGOVERN, San Diego, CA (US); Longquan CHEN, Lexington, MA (US); Bhavik JAVIA, Delhi (IN)

(73) Assignees: Boston Scientific Scimed, Inc., Maple Grove, MN (US); Boston Scientific Medical Device Limited, Galway (IE)

(21) Appl. No.: 19/656,013

(22) Filed: Apr. 23, 2026

Related U.S. Application Data
(60) Provisional application No. 63/797,230, filed on Apr. 30, 2025.

(51) Int. Cl. G16H 40/63; G16H 10/60; G16H 40/20

(52) U.S. Cl. CPC G16H 40/63; G16H 10/60; G16H 40/20

(57) ABSTRACT

Processing of medical system requests, including voice requests, are described. For example, a recording is received that includes a voice request captured by a medical system during a medical procedure, and audio content of the recording is transcribed to text. A response to the voice request is generated by: determining, based on the text, to invoke a tool from a plurality of tools, the tool having a function associated with the voice request; responsive to a call to the tool, receiving a result generated from an execution of the function by the tool based on inputs provided as part of the call, the inputs including at least a portion of the text; and generating the response to the voice request based on the text and the result. The response is provided to the medical system for presentation on a display of the medical system.

### CROSS-REFERENCE TO RELATED APPLICATIONS

This application claims the benefit of priority from U.S. Provisional Application No. 63/797,230, filed on Apr. 30, 2025, which is incorporated by reference herein in its entirety.

### TECHNICAL FIELD

The disclosure relates generally to systems and methods for medical system request processing. More specifically, aspects of the disclosure pertain to systems and methods for processing requests, including voice requests, captured by a medical system during a medical procedure, where artificial intelligence (AI) is leveraged to facilitate accurate response generation in real time.

### BACKGROUND

Endoscopic medical procedures involve the use of an endoscopic system, including an endoscope removably connected to a computing system. For example, the endoscope may be inserted into and navigated through a body lumen of a patient to a target site during a medical procedure. The endoscope may include an imaging device configured to capture images, as well as a lighting device configured to emit light to facilitate visualization of anatomical features or other objects within the lumen that are captured by the images. The computing system may control various operating parameters of the endoscope, as well as process and display the captured images received from the endoscope for viewing by an operator (e.g., a physician). Many endoscopic medical procedures are complex. During these procedures, an operator may need to quickly access information relating to use of the endoscopic system, adjust operating parameters or other settings of components of the endoscopic system, or obtain video or other medical content to provide real-time assistance or guidance to the operator in a manner that limits interruption to the medical procedure. Additionally, the operator may need to generate and store procedure notes that provide details about the medical procedure in a patient's electronic health record.

### SUMMARY

According to some aspects, the techniques described herein relate to a method for voice request processing. An example method includes: receiving a recording including a voice request captured by a medical system during a medical procedure; transcribing audio content of the recording to text; generating a response to the voice request by: determining, based on the text, to invoke a tool from a plurality of tools, the tool having a function associated with the voice request; responsive to a call to the tool, receiving a result generated from an execution of the function by the tool based on inputs provided as part of the call, the inputs including at least a portion of the text; and generating the response to the voice request based on the text and the result; and providing the response to the medical system for presentation on a display of the medical system.

In some examples, the response is generated using a large language model (LLM), and generating the response to the voice request may include: in response to receiving the text as part of a prompt, generating and including, by the LLM, code in an initial output of the LLM, the code indicating the tool to be invoked; calling, by an agent, the tool based on the code included in the initial output of the LLM; in response to receiving the result, augmenting, by the agent, the prompt to include the result to generate an augmented prompt for provision to the LLM; and processing, by the LLM, the augmented prompt to generate the response to the voice request. In further examples, the response is generated using a large language model (LLM) fine-tuned to determine the tool, from the plurality of tools, to be invoked based on one or more tokens identified in the text that are associated with the tool.

In some aspects, the voice request is to control an operating parameter of a component of the medical system, the function of the tool is associated with generating a control command for the operating parameter, and generating the response may include: receiving the result, including the control command having a syntax specific to the medical system; and including the control command in the response, where, when the response is provided to the medical system, the medical system is configured to read and execute the control command based on the syntax. For example, the medical system may include an endoscope having an imaging device and a lighting device and a computing system that the endoscope is removably connected to; the operating parameter may be an operating parameter of one of the imaging device or the lighting device; and the syntax of the control command is specific to the computing system such that, upon receiving the response, the computing system is configured to generate and transmit one or more control signals to the one of the imaging device or the lighting device to control the operating parameter.

In other aspects, the voice request is a request to retrieve a video associated with an aspect of the medical procedure for playback on a display component of the medical system during the medical procedure, and the function of the tool is associated with interacting with a video retrieval system. The video retrieval system may be configured to: transform the text to generate transformed text in a format compatible with a video search data store, the video search data store configured to store, for each video of a plurality of videos, one or more transformed portions of video transcript corresponding to one or more portions of video data of the video; identify, from the video search data store and based on the transformed text, a transformed portion of video transcript relevant to the transformed text, the transformed portion of video transcript being associated with the aspect of the medical procedure; and generate, as the result, a portion of video transcript corresponding to the transformed portion of video transcript, a timestamp associated with the portion of video transcript, and video data associated with the portion of video transcript, where the response includes at least the timestamp and the video data and, upon receiving the response, the medical system plays back the video data at a time corresponding to the timestamp. In some examples, the response further includes the portion of video transcript, and the medical system displays the portion of video transcript in addition to the playback of the video data at the time corresponding to the timestamp.

Additionally, the video retrieval system may be further configured to establish the video search data store by, for each video of the plurality of videos: receiving video data of the video; generating a video transcript from the video data; extracting metadata associated with the video data, including a plurality of timestamps; storing the video data, the video transcript, and the metadata in a video data store, where the plurality of timestamps is stored in association with a corresponding plurality of portions of the video transcript; transforming one or more of the plurality of portions of the video transcript to generate the one or more transformed portions of the video transcript; and storing the one or more transformed portions of the video transcript in the video search data store.

In some examples, the video retrieval system may further configured to: identify, from a medical search data store and based on the transformed text, a transformed portion of medical data relevant to the transformed text, the transformed portion of medical data being associated with the aspect of the medical procedure; determining a relevancy of each of the transformed portion of video transcript and the transformed portion of medical data to the request; and based on the relevancy, determining to include at least the portion of video transcript corresponding to the transformed portion of video transcript in the result. Based on the relevancy, the transformed portion of medical data may further be included in the result.

In further aspects, the response is generated using a large language model (LLM) implementing retrieval augmentation generation (RAG), and generating the response to the voice request includes: obtaining, from one or more contextual data stores, additional information for use in generating the response. In some examples, one of the one or more contextual data stores is a direction for use (DFU) data store configured to store one or more documents associated with use of the medical system, the voice request is a query related to use of a component of the medical system, and generating the response to the voice request includes: identifying, from the one or more documents within the DFU data store, information associated with the use of the component of the medical system; and including at least a portion of the information in the response.

In yet further aspects, the recording including the voice request is a first recording, the voice request is to record and transcribe the medical procedure, the response includes a command that is read and executed by the medical system to cause the medical system to generate a second recording of the medical procedure, and the method further includes: receiving the second recording; transcribing audio content of the second recording to text to generate a medical procedure transcript; and automatically storing the medical procedure transcript in association with an electronic record of a patient.

In some examples, the medical system includes a microphone, and the voice request is captured and converted, by the microphone, to electrical signals including the recording.

According to other aspects, the techniques described herein relate to a method for request processing. An example method includes: receiving a request, captured by a medical system, to control an operating parameter of a component of the medical system during a medical procedure; generating a response to the request by: determining, based on a text of the request, to invoke a tool from a plurality of tools, the tool having a function associated with generating a control command for the operating parameter; responsive to a call to the tool, receiving a result generated from an execution of the function by the tool, the result including the control command having a syntax specific to the medical system; and generating the response to the request based on the text and the result, the response including the control command; and providing the response to the medical system, where the medical system reads and executes the control command based on the syntax.

In some examples, the request is a voice request, and the method further includes: receiving a recording including the voice request captured by the medical system; and transcribing audio content of the recording to the text for use in generating the response.

In some aspects, medical system includes an endoscope having an imaging device and a lighting device and a computing system that the endoscope is removably connected to; the operating parameter is an operating parameter of one of the imaging device or the lighting device; and the syntax of the control command is specific to the computing system such that, upon receiving the response, the computing system is configured to generate and transmit one or more control signals to the one of the imaging device or the lighting device to control the operating parameter.

According to further aspects, the techniques described herein relate to a method for request processing. An example method includes: receiving a request, the request captured by a medical system, to retrieve a video associated with an aspect of a particular type of medical procedure for playback on a display component of the medical system during a medical procedure of the particular type; transforming text of the request to generate transformed text in a format compatible with a video search data store, the video search data store configured to store, for each video of a plurality of videos, one or more transformed portions of video transcript corresponding to one or more portions of video data of the video; identifying, from the video search data store and based on the transformed text, a transformed portion of video transcript relevant to the transformed text, the transformed portion of video transcript being associated with the aspect of the particular type of medical procedure; generating a response to the request that includes a portion of video transcript corresponding to the transformed portion of video transcript, a timestamp associated with the portion of video transcript, and video data associated with the portion of video transcript; and providing the response to the medical system, where the medical system plays back the video data at a time corresponding to the timestamp.

In some examples, the video search data store is established by, for each video of the plurality of videos: receiving video data of the video; generating a video transcript from the video data; extracting metadata associated with the video data, including a plurality of timestamps; storing the video data, the video transcript, and the metadata in a video data store, where the plurality of timestamps is stored in association with a corresponding plurality of portions of the video transcript; transforming one or more of the plurality of portions of the video transcript to generate the one or more transformed portions of the video transcript; and storing the one or more transformed portions of the video transcript in the video search data store.

In other examples, the method may further include: identifying, from a medical search data store and based on the transformed text, a transformed portion of medical data relevant to the transformed text, the transformed portion of medical data being associated with the aspect of the particular type of medical procedure; determining a relevancy of each of the transformed portion of video transcript and the transformed portion of medical data to the request; and based on the relevancy, determining to include at least the portion of video transcript corresponding to the transformed portion of video transcript.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “and/or” includes “or”. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10% of a stated value.

### BRIEF DESCRIPTION OF THE DRAWINGS

The accompanying drawings, which are incorporated in and constitute a part of this specification, illustrate examples of this disclosure and, together with the description, serve to explain the principles of the disclosure.

FIG. 1 depicts an example environment for medical system request processing.

FIGS. 2A and 2B depict example methods for processing voice requests.

FIG. 2C depicts an example request processing pipeline for processing voice requests in accordance with the example methods of FIGS. 2A and 2B.

FIG. 3 depicts an example method for generating responses to requests.

FIG. 4 depicts example processes for training, deploying, and monitoring a machine learning model configured to generate responses to requests.

FIG. 5A depicts an example process for establishing one or more search data stores.

FIG. 5B depicts an example process for processing a request using the one or more search data stores established using the process of FIG. 5A.

FIG. 5C depicts an example system flow diagram for establishing the one or more search data stores for subsequent use in processing the request in accordance with the processes of FIGS. 5A and 5B.

FIG. 6 depicts an example user interface.

FIG. 7 depicts an example of a computer.

### DETAILED DESCRIPTION

As briefly mentioned above, endoscopic medical procedures involving the use of an endoscope, equipped with imaging and lighting devices, and removably connected to a computing system, are often complex. During these procedures, an operator may need to quickly access information relating to use of the endoscope, adjust operating parameters or settings of the endoscope or computing system, or obtain video or other medical content to provide real-time assistance or guidance to the operator in a manner that limits interruption to the medical procedure. Additionally, the operator may need to generate and store procedure notes that provide details about the medical procedure in a patient's electronic health record.

As an operator is performing an endoscopic medical procedure, the operator is holding the endoscope and maneuvering or manipulating components of the endoscope and potentially other accessory devices, which commonly requires the use of both the operator’s hands. As a result, the operator may rely on other individuals that are in the procedure room to perform various tasks requested by the operator. For example, other individuals may manually transcribe the operator's speech throughout the medical procedure to document details of the procedure. Alternatively, the operator may wait and attempt to recall and document the procedure details after completing the medical procedure (or several additional procedures), which may lead to incomplete or inaccurate documentation.

Additionally, the operator is often a distance away from the computing system that includes controls for adjusting various operating parameters or functions associated with the endoscope or the computing system. Therefore, if the operator wants to make such adjustments during the medical procedure, it is difficult (or impossible) for the operator to do so without interrupting the procedure or requesting assistance from another individual in the procedure room.

In some scenarios, the operator may need additional information during the medical procedure, such as information related to use of a component of the endoscopic system or a reference video showing an advanced technique to be performed as the next step of the medical procedure. Conventional methods for retrieving information requested by an operator as a medical procedure is being performed are time-consuming and inefficient. For example, computing devices or systems separate from the endoscopic system (and potentially even outside of the procedure room) are often utilized by the operator or other individuals to obtain the information requested.

Additional challenges may be faced, depending on a type of information being requested. As one example, the operator may want to view a stored video of the same type of medical procedure as a reference or guide for performing a portion or aspect of the medical procedure. However, stored videos of medical procedures are often lengthy (e.g., recordings of entireties of the medical procedures), which may make it challenging to quickly locate specific segments of the video that convey the portion or aspect of interest. Similarly, an unstructured or long-form content format in which the videos are commonly stored often fails to highlight any valuable insights gained from the medical procedures (e.g., how techniques are successfully adjusted when certain anatomical anomalies were detected) that could be leveraged when searching for relevant videos during future medical procedures. Further, common video search databases are limited to keyword-based search, which provides less than accurate results, as a result of variations in medical terminologies and difficulty in applying keyword search to video data.

To address and overcome these challenges, systems and methods are described herein for artificial intelligence (AI)-assisted request processing in real time during a medical procedure, including the capability to process voice requests. To enable voice request processing, an ambient recording feature may be integrated with the endoscopic system via an addition of a communicatively connected headset or an inclusion of a microphone within the computing system, for example, to generate an audio recording that captures a hands-free voice request from the operator. Using one or more AI models that form part of a request processing pipeline, the audio may then be transcribed to text, and the text may be processed to generate a response to the request that may be visually or audibly presented back to the operator by the endoscopic system. Text requests may also be received by the endoscopic system and similarly processed via the request processing pipeline, but may bypass transcription as the requests are already in text format when received.

In some examples, the various processes performed along the request processing pipeline may be executed by one or more local machines, such as the computing system of the endoscopic system, one or more edge devices, or a combination of the computing system and edge devices. In other examples, at least a portion of the processes performed along the request processing pipeline may be executed remotely by one or more server-side systems.

The requests may include queries, such as a question relating to use of a component of the endoscopic system, or issued commands, such as a command to control or adjust an operating parameter or setting of the endoscopic system or a command to retrieve video or other medical content for presentation to assist or guide the operator. A request processing system within the request processing pipeline may include a large language model (LLM) configured to process the request to generate a response.

In sone examples, the LLM may be a Retrieval Augmented Generation (RAG)-enhanced LLM that implements RAG techniques to generate more accurate, contextually relevant responses. For example, when the request is a query related to use of an endoscopic system component, RAG techniques may be used to identify relevant information from a contextually relevant data store, such as a Direction for Use (DFU) data store storing documents defining use of the various components of the endoscopic system.

Additionally or alternatively, the LLM may be a component of an agent framework with tool-calling functionality to enable retrieval of data, performance of specific actions or tasks, and/or interactions with additional systems to obtain information beyond a knowledge base of the LLM for use in generating responses. As one example, at least a subset of the available tools may be associated with functions for controlling aspects of the endoscopic system that may be called when the request includes a command to control or adjust an operating parameter or setting of the endoscopic system. When called, these functions may return, as a result, commands having a syntax specific to the endoscopic system. For example, the commands may include a specific structure and format understood and used by the endoscopic system to perform the operation or adjustment requested. Accordingly, the commands are executable by the endoscopic system upon receipt of a response generated to include the syntax-specific commands.

As another example, at least another available tool may facilitate the retrieval and playback of relevant video segments in real time from training or historical cases, or surfacing of related procedural content from trusted web sources in response to a request for such information during the procedure to provide guidance or assistance to the operator. For example, the tool may enable interaction with a video retrieval system. The video retrieval system may establish a video search data store, and optionally a medical search data store, in a manner to facilitate improved search. For example, the search data stores may be established to facilitate semantic similarity searches or graph-based searches as opposed to keyword-based searches. Additionally, the video data or optional medical data may be transformed for storage within the respective search data stores in a structured format that highlights valuable insights gained from the medical procedures. For example, if a segment of video included how a technique was successfully adjusted when an anatomical anomaly was detected, the video segment can be stored in a manner such that it is labeled or otherwise associated with (e.g., via metadata) the technique and the anomaly. That way, if an operator in a future medical procedure encounters the same anomaly and requests content related to how to perform the technique when the anomaly is present, the video segment can be quickly identified and retrieved.

In other aspects, vision language models (VLMs) may be implemented to generate searchable videos. In other words, videos may be transformed into a searchable format by applying a VLM to video data of the videos to generate a video transcript. For example, the VLM may process video data of a video of a medical procedure in real-time to generate, as the video transcript, text that describes what is occurring throughout the video based on visual information extracted from the video data. The video transcript may then be converted into a structured format for storage.

Separate from, or in addition to, the request processing, systems and methods are described for medical procedure transcription. For example, responsive to a voice or text request processed as described above, or an alternative enabling of a transcription feature of the endoscopic system, an audio recording of the medical procedure may be captured, transcribed, and automatically stored in association with a patient's electronic heath record. Additionally, or alternatively, a video recording of the medial procedure may be captured, transcribed (e.g., using one or more VLMs), and automatically stored in association with the patient’s electronic health record. In some examples, to help train the VLM, or retrain the VLM to improve an accuracy thereof, the text generated and output by the VLM may be compared to a transcript generated from a simultaneously captured audio recording.

Timestamps from the audio recording or the video recording may be extracted from and stored in association with the transcript. In some examples, images or videos recorded and saved during the medical procedure may also be automatically stored in association with a patient's electronic heath record, and have associated timestamps. In such examples, the timestamps may be used to associate a portion of the transcript with the image or video recorded. For example, if one of the images shows an area of interest, a portion of the transcript including the operator's observations regarding that area of interest may be identified using the timestamps and stored in association with (e.g., as an annotation to) the image.

Specific examples described herein implement an endoscopic system and associated request processing pipeline configured to perform request processing. However, it should be understood that techniques described herein may be adapted to other medical systems. It should also be understood that the examples above are illustrative only. The techniques and technologies described herein may be adapted to any suitable activity.

Additionally, presented below are various aspects of AI or machine learning techniques that may be adapted to facilitate the request processing. As will be discussed in more detail below, these AI or machine learning techniques may include, e.g., a particular selection of training data, a particular training process for a model, operation of a particular device or system suitable for use with the trained model, operation of the model in conjunction with particular data, modification of such particular data by the model, etc., or other aspects that may be apparent to one of ordinary skill in the art based on the description herein.

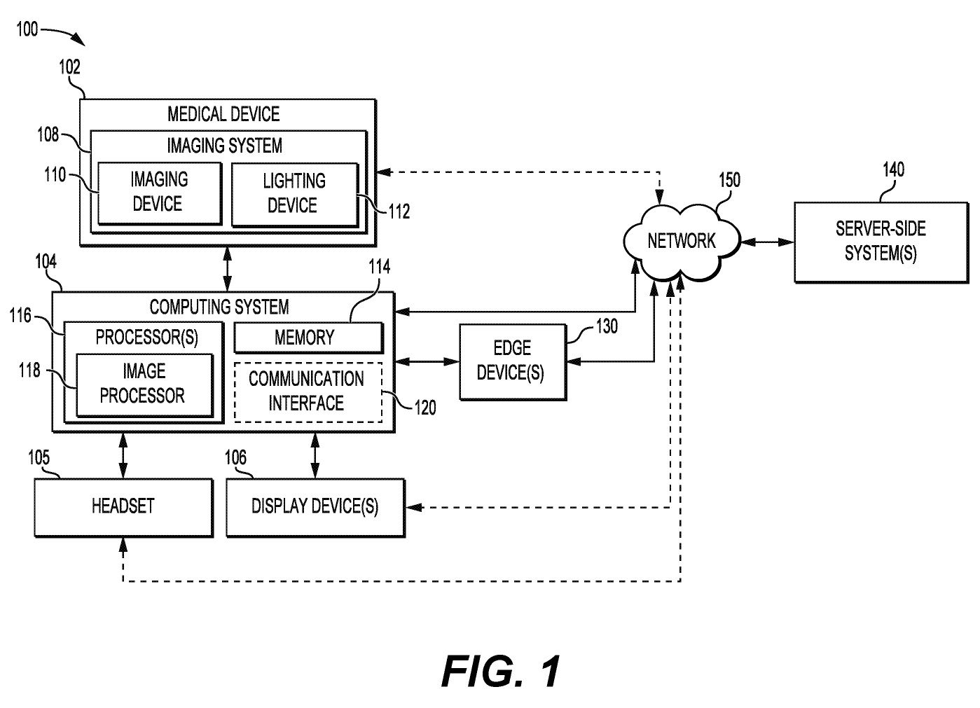

FIG. 1 depicts an example environment for medical system request processing. A medical system 100 may include a medical device 102, a computing system 104, a headset 105, one or more display device(s) 106, and one or more edge device(s) 130. In some examples, medical system 100 may communicate with one or more server-side system(s) 140 over a network 150.

Medical device 102 may be used to perform a diagnostic and/or interventional medical procedure on a patient upon a removable connection with computing system 104. Medical device 102 may be an endoscope or other type of scope, such as a bronchoscope, ureteroscope, duodenoscope, cholangioscope, gastroscope, endoscopic ultrasonography ("EUS") scope, colonoscope, laparoscope, arthroscope, cystoscope, aspiration scope, sheath, or catheter, among other examples.

In some examples, medical device 102 may be a single use device that is discarded or disposed of upon disconnection from computing system 104 (e.g., at an end of the medical procedure). Therefore, although only one of medical device 102 is shown in FIG. 1, medical system 100 may include one or more other medical devices 102 that are the same or similar to medical device 102, and may also be removably connected to computing system 104 for performing medical procedures. Additionally, in some examples, different types of medical devices may be connectable to computing system 104 simultaneously with or independently from medical device 102.

Medical device 102 may include an imaging system 108. Imaging system 108 may include at least one imaging device 110 and at least one lighting device 112. Imaging device 110 may be located at a distal end of medical device 102 (e.g., at a distal tip of medical device 102). Imaging device 110 may be configured to continuously capture image signals as the distal end of medical device 102 is inserted into and navigated through a body lumen of the patient to a target site during the medical procedure. Imaging device 110 may include one or more cameras, one or more image sensors, or one or more endoscopic viewing elements, among other similar devices.

In some examples, lighting device 112 may be located at the distal end of medical device 102 (e.g., at the distal tip of medical device 102) along with imaging device 110. In other examples (not shown), lighting device 112 may be a separate device or may be integrated with computing system 104, with light from lighting device 112 being transmitted via fibers (e.g., optical fibers) extending a length of medical device 102 (e.g., from a proximal end of medical device 102 connected to computing system 104 to the distal tip of medical device 102). Lighting device 112 may be configured to illuminate areas of the patient's body (e.g., the target area) during the medical procedure to facilitate imaging of the target area by imaging device 110. Lighting device 112 may include one or more LEDs, incandescent light sources, optical fibers (e.g., optical fibers to transmit a light from a proximal light source), and/or other illuminators.

One or more components of medical device 102, including imaging system 108 and the components thereof, may be communicatively coupled to computing system 104 via wired connections and/or wireless connections (e.g., over network 150) to enable communication of various signals between medical device 102 and computing system 104. For example, image signals captured by imaging device 110 (e.g., raw image data) may be received by computing system 104. Additionally, computing system 104 may provide one or more signals to the imaging device 110 and/or lighting device 112 to cause one or more operating parameters of imaging device 110 and/or lighting device 112, respectively, to be adjusted. In some examples, and as described in more detail below, the adjustments may be made responsive to requests provided to computing system 104 by an operator.

In some examples, computing system 104 is a controller, a control unit, a computing device, or other similar standalone processing unit separate from medical device 102. In other examples, computing system 104 may be partially or fully integrated with medical device 102. Computing system 104 may be or at least include a field-programmable gate array (FPGA) comprising programmable logic blocks for performing various functions, including at least a portion of request processing steps.

Computing system 104 may include at least one memory 114, one or more processor(s) 116, and at least one communication interface 120. In some examples, memory 114, processor(s) 116, and communication interface 120, may be embedded on a printed circuit board (PCB) forming a system-on-module (SOM) of computing system 104.

Memory 114 may store instructions to be executed by processor(s) 116 to cause computing system 104 to perform corresponding operations. At least a portion of the instructions stored in memory 114 may include instructions for processing requests, including both text and voice requests. Additionally, one or more electronic application(s), e.g., a program, plugin, browser extension, etc., may be installed on memory 114. One example electronic application may be a chatbot application configured to receive and respond to requests. In some embodiments, the chatbot application may be associated with or otherwise enable communication with one or more of the other components in the environment, such as edge device(s) 130 or server-side system(s) 140. In some examples, the chatbot application may be a thick client application that is locally installed on computing system 104. In other examples, the chatbot application may be a thin client application (e.g., a web application) that is rendered via a web browser launched on computing system 104.

Memory 114 may also store any models, algorithms, or other processes that are executable to perform the operations. For example, in some aspects, AI models, including a speech-to-text (STT) model configured to transcribe audio to text or a large language model (LLM) configured to generate a response to a request, may be stored in memory 114 and executed locally. In other examples, one or both of these models may be stored and executed by other components locally or remotely to conserve memory and processing resources of computing system 104, as described in detail below. Memory 114 may also include one or more data stores. Additionally or alternatively, computing system 104 may include one or more data stores separate from memory 114. The data stores may, at least temporarily, store data associated with the requests.

Processor(s) 116 may include at least one image processor 118. Image processor 118 may be configured to process one or more image signals (e.g., raw image data) captured by imaging device 110 and received by computing system 104 to generate an image. In some examples, image processor 118 may be or include an FPGA, a digital signal processing (DSP) processor, a graphics processing unit (GPU), or the like.

Processor(s) 116 may also perform at least some steps or operations associated with the request processing independently or, dependent on the request, in conjunction with image processor 118. In some examples, other steps or operations associated with the request processing may be performed by one or more other components of the environment, such as edge device(s) 130 or server-side system(s) 140.

Edge device(s) 130 may be local devices communicatively coupled to computing system 104 and network 150 to facilitate data transmission between computing system 104 and network 150. Edge device(s) 130 may include controllers, control units, computing devices, or other similar standalone processing units. In some aspects, edge device(s) 130 may include at least one AI edge device configured to support AI processing. The AI edge device may be capable of executing resource intensive AI models, such as the STT model or LLM. In some examples, edge device(s) 130 may perform one or more request processing steps in conjunction with computing system 104 locally. This configuration may allow for distributed request processing, which may reduce the computational load on computing system 104 while maintaining low latency.

Additionally or alternatively, server-side system(s) 140 may include one or more systems configured to perform one or more request processing steps remotely, as described in more detail with reference to 2A-2C below. In such examples, the AI models, such as the STT model or LLM, may be stored and executed remotely by one or more of server-side system(s) 140.

In some examples, the request processing, including text request processing and voice request processing, may be provided as an assistant feature of computing system 104 that can be enabled and disabled (e.g., turned on and off) by the operator. For example, graphical user interface control elements generated and provided for display by computing system 104 or buttons on medical device 102 may be actionable to turn the assistant feature on and off.

Communication interface 120 of computing system 104 may provide connectivity to network 150. Communication interface 120 may also provide connectivity to medical device 102, headset 105, or display device(s) 106. In some examples, a communicative connection between computing system 104 and medical device 102 (or components thereof) or between computing system 104, headset 105, and display device(s) 106 may be at least partially supported via network 150.

Although only one computing system 104 is shown in FIG. 1, in some examples, medical system 100 may include one or more other computing systems 104 that are the same or similar to computing system 104 to which medical device 102 may also be removably connected to, as described below. To provide an illustrative example, within a medical facility, multiple procedural suites may each include one computing system 104 that remains located in that suite.

Headset 105 may be communicatively coupled to computing system 104. In some examples, headset 105 may be a universal serial bus (USB) headset or a Bluetooth^{®} headset. Although the term “headset” is used herein, headset 105 may include earbuds, headphones, a microphone, or other similar devices. Headset 105 may include a microphone configured to capture and convert voice (e.g., speech) into electrical signals that are transmitted to computing system 104 for storage as an audio file. In some aspects, there may be graphical control elements on a graphical user interface of one of display device(s) 106 or control buttons on headset 105 that, upon actuation, may cause the microphone to begin capturing and converting voice into electrical signals for transmission to computing system 104. In other aspects, a lightweight audio classifier may be continuously run that listens for a wake word spoken by the operator. Additionally, or alternatively, computing system 104 may include a microphone configured to perform similar operations as the microphone of headset 105.

Display device(s) 106 may be configured to display image data, including at least the image generated by computing system 104. Display device(s) 106 may include one or more a combination of monitors, computing device screens, touch screen display devices, etc. In some examples, one or more of the display device(s) 106 may be a separate device from computing system 104 that is communicatively coupleable to computing system 104 via wired and/or wireless connections. In other examples, at least one of display device(s) 106 may be a display of computing system 104 itself.

In some examples, computing system 104 may generate, or may cause to be generated, one or more graphical user interfaces based on instructions or information stored in memory 114, instructions or information received from one or more server-side system(s) 140, and/or the like and may cause the graphical user interfaces to be displayed via display device(s) 106. The graphical user interfaces may be, e.g., application interfaces or browser user interfaces and may include text, selection controls, and/or the like, in addition to the displayed image data. One example application interface displayed may be a chatbot application interface of the local or web-based chatbot application executing on computing system 104.

Display device(s) 106 may include a touch screen or a display with other input systems (e.g., a mouse, keyboard, voice, etc.) for an operator of computing system 104 to control functions of computing system 104 or functions of other components via computing system 104, such as functions of medical device 102, headset 105, or display device(s) 106. As one example, the operator may select one or more of the control elements displayed on a graphical user interface of display device(s) 106 to enable (e.g., turn on) one or more features, such as the assistant feature or a transcription feature. As another example, the operator may select one or more of the control elements displayed on a graphical user interface of display device(s) 106 to cause an adjustment of one or more operating parameters of imaging system 108 (e.g., based on operator preferences). In other examples, when the assistant feature is enabled, the operator may provide a voice or text request to cause an adjustment of the parameters. The respective selection or a response generated from a processing of the request may be received by computing system 104 and cause corresponding signals to be transmitted from computing system 104 to imaging system 108 and/or specific components thereof. Similar requests may be provided and processed to cause performance of other functions controllable by computing system 104, such as image-enhancement features of computing system 104.

One or more components of medical system 100, such as medical device 102, computing system 104, headset 105, or display device(s) 106, may be capable of network connectivity, and may communicate with one another over a wired or wireless network, such as network 150. Network 150 may be an electronic network. Network 150 may include one or more wired and/or wireless networks, such as a wide area network ("WAN"), a local area network ("LAN"), personal area network ("PAN"), a cellular network (e.g., a 3G network, a 4G network, a 5G network, etc.), or the like. In other examples, the components of medical system 100 may communicate and/or connect to network 150 over USB or other similar local, low latency connections or direct wireless protocol. Components of medical system 100 may be connected via network 150, using one or more standard communication protocols, such that the component may transmit and receive communications from each other across network 150.

In some examples, one or more of the components of medical system 100 communicate with one or more server-side system(s) 140 over network 150. In some examples, one or more of server-side system(s) 140 may be configured to generate, train, or deploy one or more of the AI models, such as the voice-to text model or LLM, that may be used individually or in combination for request processing (e.g., dependent on if the request is a text or voice request). In some examples, the same and/or different server-side system(s) 140 may include remote image processing systems configured to optionally perform certain types of image processing, including but not limited, more resource intensive AI image processing, if being utilized, e.g., to conserve local resources of computing system 104 when network connectivity is available).

Additionally or alternatively, server-side system(s) 140 may include remote data storage systems for storing data generated by one or more components of the environment (e.g., image data, video data, or data generated as a result of request processing, such as conversations including requests and responses, medical procedure recordings and transcripts, feedback data, etc.). In some examples, at least one of the data storage systems may include a picture archiving and communication system (PACS) that stores the image or video, along with other types of imaging data from various imaging modalities (e.g., ultrasound, magnetic resonance, nuclear medicine imaging, positron emission tomography, computed tomography, mammograms, digital radiography, histopathology, etc.) associated with the patient. PACS may also store transcripts representing operator speech captured in association with an image or video. For example, any converted speech-to-text, as well as images or videos captured during the medical procedure may be timestamped and wrapped together (e.g., with the text as metadata) into a DICOM file that is transmitted to the PACs for storage.

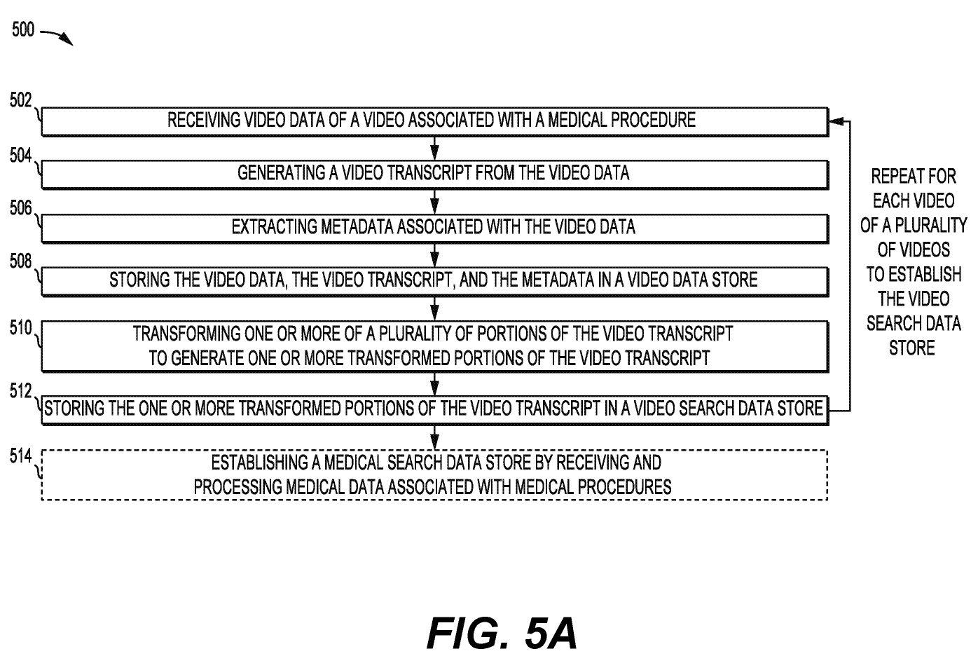

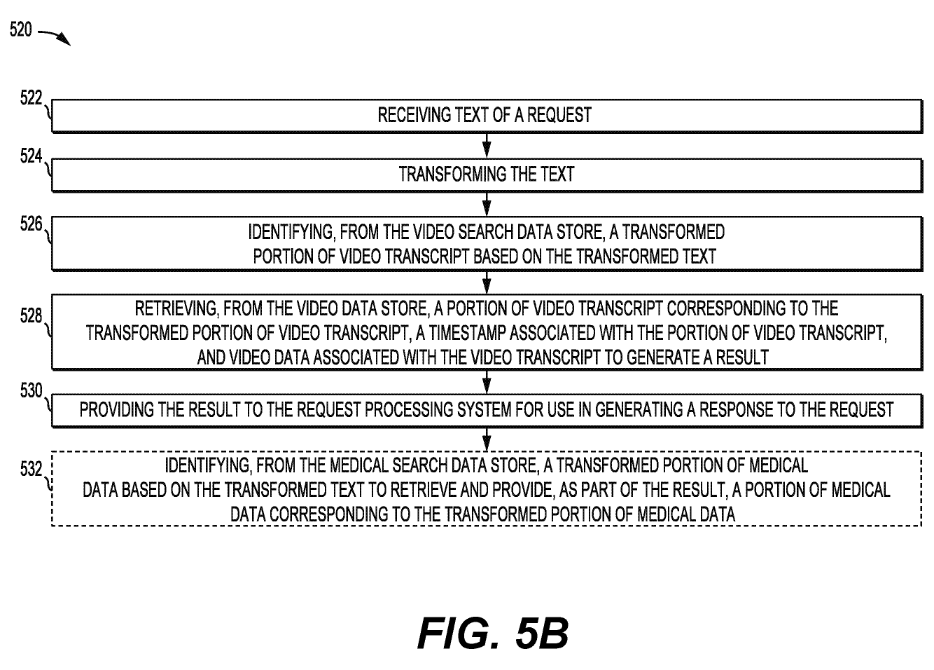

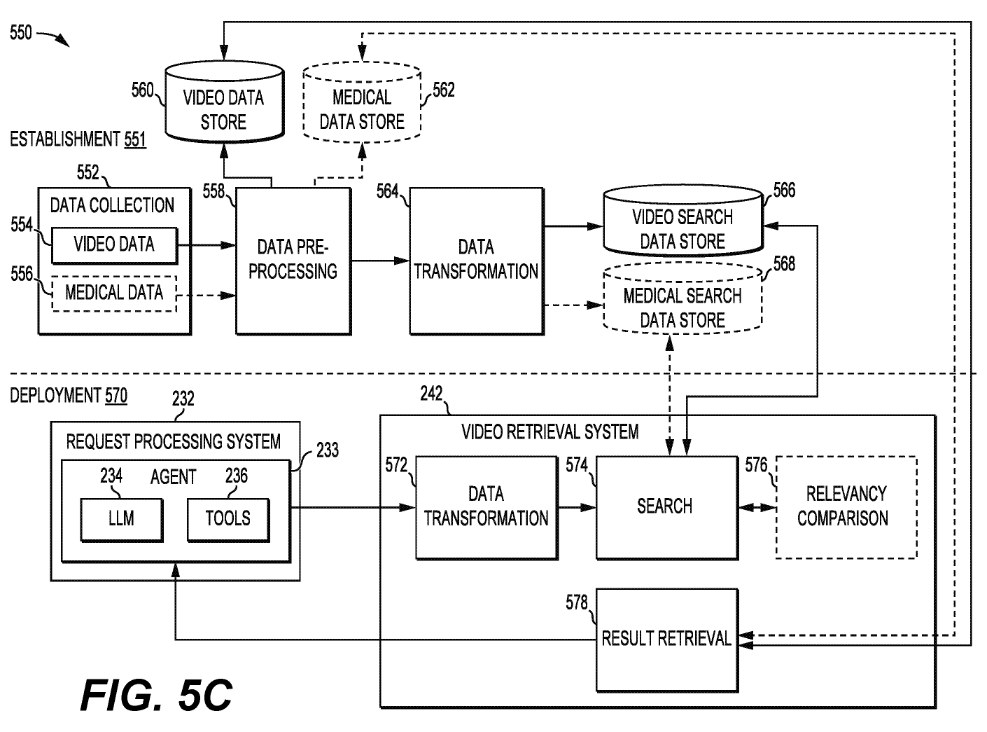

In further examples, at least one of the data storage systems may include one or more searchable or queryable data stores (e.g., search data stores) configured to store contextual data for use in generating the response. The search data stores may include, but are not limited to, a vector store or an embedding store. As one example, a Direction For Use (DFU) data store may be configured to store DFU documents for at least the various systems, devices, accessories, or components of medical system 100. The DFU data store may be searchable (e.g., via keyword or semantic search) to obtain instructions for use or other similar troubleshooting information responsive to a request. As another example, a video search data store may be searchable to identify a relevant segment of a video responsive to a request. Similarly, a medical search data store may optionally be searchable to identify a relevant portion of medical content or information responsive to a request to supplement or provide independently of the relevant segment of video. In some examples, and as described in more detail with reference to FIGS. 5A-5C, separate data stores from the searchable data stores may respectively store the video data and medical data that is identifiable via the search data stores. For example, a transformed version of the video and medical data may be included within the searchable data stores to facilitate search.

Although various components in medical system 100 are depicted as separate components in FIG. 1, it should be understood that a component or portion of a component in medical system 100 may, in some examples, be fully or partially integrated with or incorporated into one or more other components. For example, one of display device(s) 106 may be integrated with computing system 104 and/or computing system 104 may be integrated with medical device 102. In some aspects, operations or aspects of one or more of the components discussed above may be distributed amongst one or more other components. Any suitable arrangement and/or integration of the various systems and devices of medical system 100 may be used.

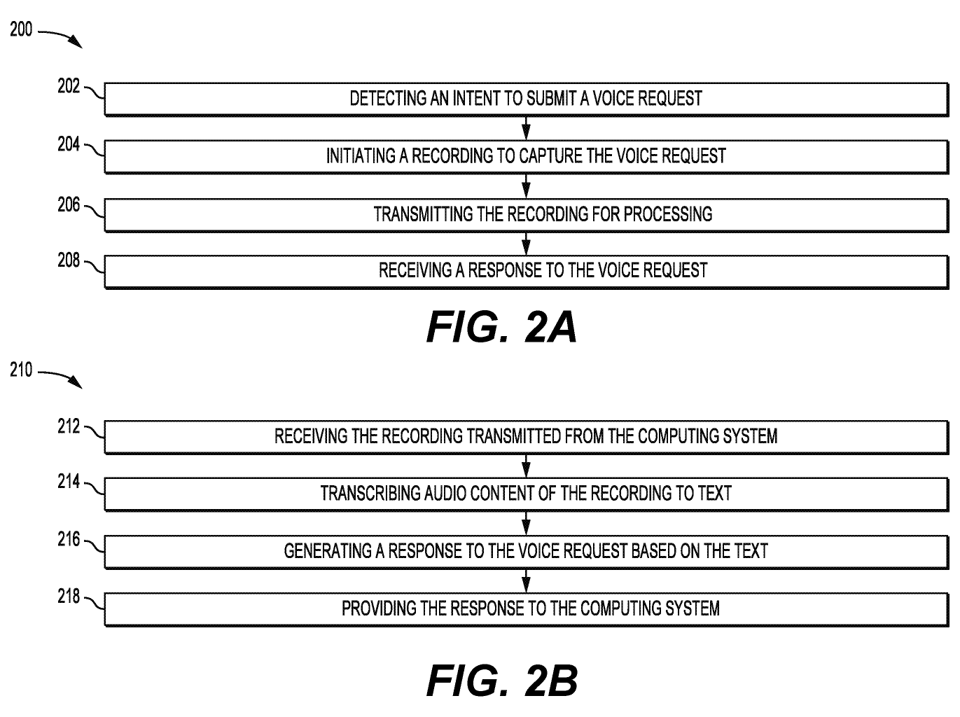

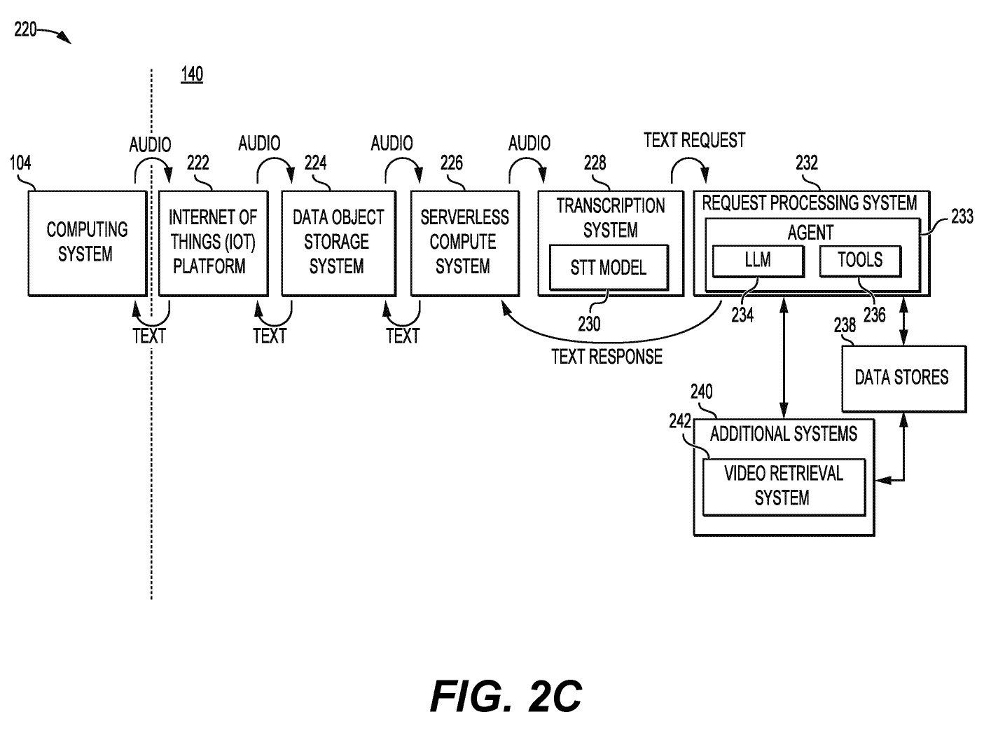

FIGS. 2A and 2B depict example methods 200, 210 for processing voice requests when at least a partially remote request processing pipeline 220 is leveraged, as shown in FIG. 2C. Specifically, FIG. 2A depicts method 200 performed by computing system 104, and FIG. 2B depicts method 210 performed by at least a subset of server-side system(s) 140.

Referring to FIGS. 2A and 2C concurrently, at step 202, method 200 may include detecting an intent to submit a voice request. In some examples, an operator may provide input via a touchscreen display of computing system 104 (e.g., one of display device(s) 106) to indicate an intent to ask a question or issue a command using the assistant feature of computing system 104. In some examples, the input may include launching or otherwise activating a chatbot application executing locally or via a web browser executing on computing system 104. In other examples, computing system 104 may passively listen, via a lightweight audio classifier, for a wake word spoken by the operator that indicates a voice request is about to be submitted. In such examples, upon receipt of the wake word, the chatbot application may be automatically launched or otherwise activated.

At step 204, method 200 may include initiating a recording to capture the voice request. In some examples, the operator may speak the voice request into the microphone of headset 105. The microphone of headset 105 may capture and convert the operator's speech into electrical signals that are transmitted to computing system 104 and stored in an audio data format (e.g., as an audio file) in memory 114. In other examples, computing system 104 may include a microphone that captures and converts the operator's speech into electrical signals for storage as an audio file in memory 114.

At step 206, method 200 may include transmitting the recording for processing. The recording may be transmitted in the audio data format (e.g., as an audio file). As one example, computing system 104 may automatically initiate an upload of the recording to the server-side portion of request processing pipeline 220. Specifically, and as shown in FIG. 2C, computing system 104 may upload the recording via an Internet of Things (IoT) platform 222. IoT platform 222 may be configured to, among other things, facilitate transfer of data between computing system 104 and the cloud, where server-side system(s) 140 reside. The data transfer may include both uploading of data from computing system 104 to the cloud and downloading of data from the cloud to computing system 104. In response to computing system 104 initiating the upload, IoT platform 222 returns, to computing system 104, a uniform resource locator (URL) to a location within a data object storage system 224 where the recording will reside within the cloud. Computing system 104 may then complete the upload via the URL to store the recording at the location within data object storage system 224.

In some examples, prior to the transmitting of the recording, the operator can specify a file name for the recording. An identifier of computing system 104 known to IoT platform 222, the file name, or both may be used to help track data associated with the recording throughout request processing pipeline 220.

Turning to FIG. 2B, and now referring concurrently to FIGS. 2B and 2C, at step 212, method 210 may include receiving the recording transmitted from computing system 104. Serverless compute system 226 monitors data object storage system 224. Whenever there is a new file uploaded for storage, serverless compute system 226 may invoke functions associated with a type of the new file received and stored. For example, the upload of the recording to data object storage system 224 may trigger serverless compute system 226 to invoke a STT model 230 associated with a transcription system 228, and pass the recording to STT model 230 for processing.

At step 214, method 210 includes transcribing audio content of the recording to text. Transcription system 228 may be an endpoint deploying STT model 230. STT model 230 may be configured to transcribe the audio content of the recording to text. The text may be passed back to serverless compute system 226.

In some aspects, STT model 230 may be a pretrained or off-the-shelf model, such as the Whisper large-v3 model, configured for speech recognition and translation. Optionally, a dictionary may be provided to the pretrained or off-the-shelf model that includes medical or endoscopic terminology to facilitate the speech recognition and translation within the environment depicted in FIG. 1. The medical terminology may include names of systems, devices, or products, as well as anatomical terms, procedural terms, and the like. In other aspects, the pretrained or off-the-shelf model may be fine-tuned using training datasets that include audio recordings with medical or endoscopic terminology in the audio content, and ground truth transcripts of the audio recordings.

Additionally, as STT model 230 is being deployed, transcripts output from STT model 230 may be monitored. In some examples, STT model 230 may output a confidence score (e.g., a predicted accuracy) associated with the transcript. If the confidence score is below a predefined threshold, the transcript may be stored along with the corresponding audio recording, and a corrected transcript (e.g., an accurate transcript) may be generated for the audio recording. The corrected transcript may be generated autonomously using a plurality of AI models (e.g., an ensemble of models trained in different domains or accents) or by a human subject matter expert. The corrected transcript may serve as a ground truth transcript for the audio recording, and may be included within a dataset with the audio recording to form a new training dataset for use in further fine-tuning or adapting STT model 230. Additionally, or alternatively, an operator feedback loop may be established as part of the monitoring. In some aspects, the operator feedback loop may particularly be leveraged when the voice request is a command to record and generate a transcript for the medical procedure (e.g., a request to enable a transcription feature). For example, in some aspects, at least a portion of the transcript may be presented via a user interface displayed on one of display device(s) 106. In such aspects, the user interface may include feedback control elements that enable the operator to indicate an accuracy of the feedback that is collected and used to generate new training datasets.

In some examples, transcription system 228 may be configured to operate independently from other server- side system(s) 140 of request processing pipeline 220. To provide an illustrative example, computing system 104 may have the transcription feature (e.g., separate from the assistant feature) that can be enabled and disabled. When enabled, audio recorded throughout an entirety of the medical procedure (or at least until the transcription feature is disabled), via a microphone included in headset 105, computing system 104, or both, may be transcribed into text and provided back to computing system 104 for display via one of display device(s) 106 in real-time. Additionally or alternatively, at an end of a recording session or at pre-defined intervals during the recording session, the transcribed text may be automatically stored in an electronic health record associated with the patient or a PACs system, for example.

At step 216, method 210 may include generating a response to the voice request based on the text. An LLM 234 may be used to generate the response. For example, once the text has been generated by transcription system 228 and passed back to serverless compute system 226, serverless compute system 226 may invoke LLM 234 associated with a request processing system 232, and pass the text (e.g., the request in a text format or "text request") to LLM 234 for processing. In some aspects, request processing system 232 may be an endpoint deploying LLM 234.

In some examples, LLM 234 may be a Retrieval Augmented Generation (RAG)-enhanced LLM that implements RAG techniques to generate more accurate, contextually relevant responses. Additionally or alternatively, LLM 234 may be a component of an agent framework with tool calling functionality. For example, request processing system 232 may include an agent 233 that, to process requests and generate responses, leverages LLM 234 and tools 236 configured to performs specific tasks or functions. The tool calling functionality may enable calling of external application programming interfaces (APIs) or other agents to perform specific tasks or functions. The agent framework may follow Model Context Protocol (MCP) or Agent2Agent (A2A) Protocol.

RAG techniques may include semantic search of one or more of a plurality of data stores 238. The data stores 238 may be configured to store contextual data in a format to facilitate semantic search to identify data or portions of data relevant to a request being processed. The identified data may then be used to generate the response.

In some aspects, vector-based semantic search may be utilized. In such aspects, the text request may be transformed into a query vector embedding. The query vector embedding may be a numerical representation of an intent and meaning of the text request. In such examples, data stores 238 may be vector databases. The vector databases may store a plurality of vector embeddings representing the contextual data in a multi-dimensional space. One or more of the vector databases may then be queried using the query vector embedding to identify one or more stored vector embeddings similar to the query vector embedding (e.g., close in distance within the multi-dimensional space). Contextual data corresponding to the identified, stored vector embeddings may then be retrieved and used by LLM 234 to generate the response. However, in some examples, vectorized data associated with intents or meanings of the text request may appear numerically similar, but those intents or meanings may be quite different when appearing as text. In other words, intent or meaning may be distorted as a result of noise introduced as part of the vector transformation process. In some aspects, applying keyword search in combination with semantic search may help to reduce effects of such distortion.

In other aspects, graph-based semantic search may be utilized. For example, a prompting technique may be applied that models a reasoning process as a graph structure. In such examples, data stores 238 may be graph databases storing one or more knowledge graphs. An example knowledge graph may represent aspects of a document comprising the contextual data as a hierarchy of nodes and relationships between the nodes as edges. In some examples, the knowledge graph may be a medical domain knowledge graph or an endoscopic domain knowledge graph that is established based on medical or endoscopic-specific documents, for example.

Example nodes within the knowledge graph may include nodes associated with a document, a section, a chunk, a table, and a row. The document node may represent an entire document (e.g., in a file format). The section nodes may represent logical sections or headings within the document that are each connected to the document node via an edge (e.g., via a HAS_Section edge). The chunk nodes may represent smaller segments or portions of text (e.g., paragraphs, lists) within a section of the document represented by one of the section modes that are used for semantic indexing. The chunk nodes may each be connected to the section node via an edge (e.g., via a UNDER_Section edge). Additionally, the chunk nodes may be sequentially connected to each other via an edge (e.g., via NEXT element edge). Further, edges may connect various nodes at the different hierarchical level to represent a hierarchical parent-child relationship (e.g., via HAS_Parent edge). Table nodes may represent table structures, if any, that are included in the document, and row nodes may represent rows of any such table structures.

To provide an illustrative implementation of RAG, during a medical procedure, an operator may submit, as a request, a question related to use of medical device 102, and specifically how to use a component of medical device 102. One of data stores 238 may include the DFU data store described with reference to FIG. 1. The DFU data store may store, among other documents, a DFU document for medical device 102 that includes instructions for use of the component. Based on the request, the DFU data stored may be searched to identify a portion of text from the document that includes the instructions for use of the component. The portion of text may be retrieved and used by LLM 234 to subsequently generate the response to the request.

When request processing system 232 leverages the agent framework, agent 233 may invoke tools 236 based on the text request to call or otherwise perform functions. The functions may be predefined functions that enable retrieval of data, performance of specific actions or tasks, and/or interactions with additional systems 240 configured to retrieve data or perform specific actions or tasks. For example, using MCP or A2A Protocol, request processing system 232 may access data stores 238 or interact with additional systems 240 (e.g., via external APIs or other agents) to obtain information beyond a knowledge base of LLM 234 for use in generating responses.

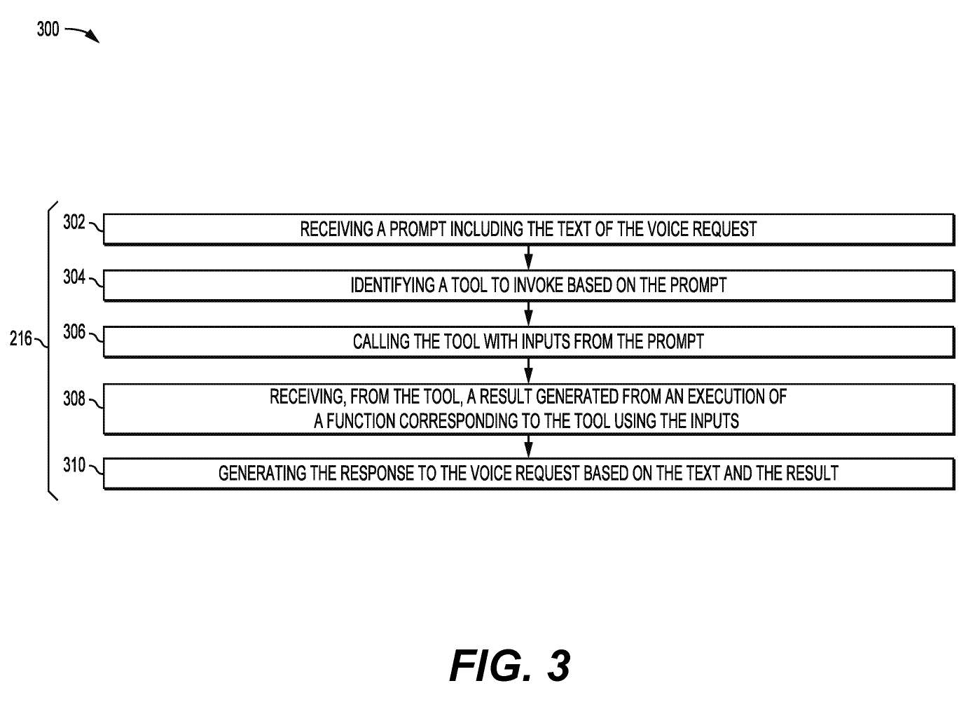

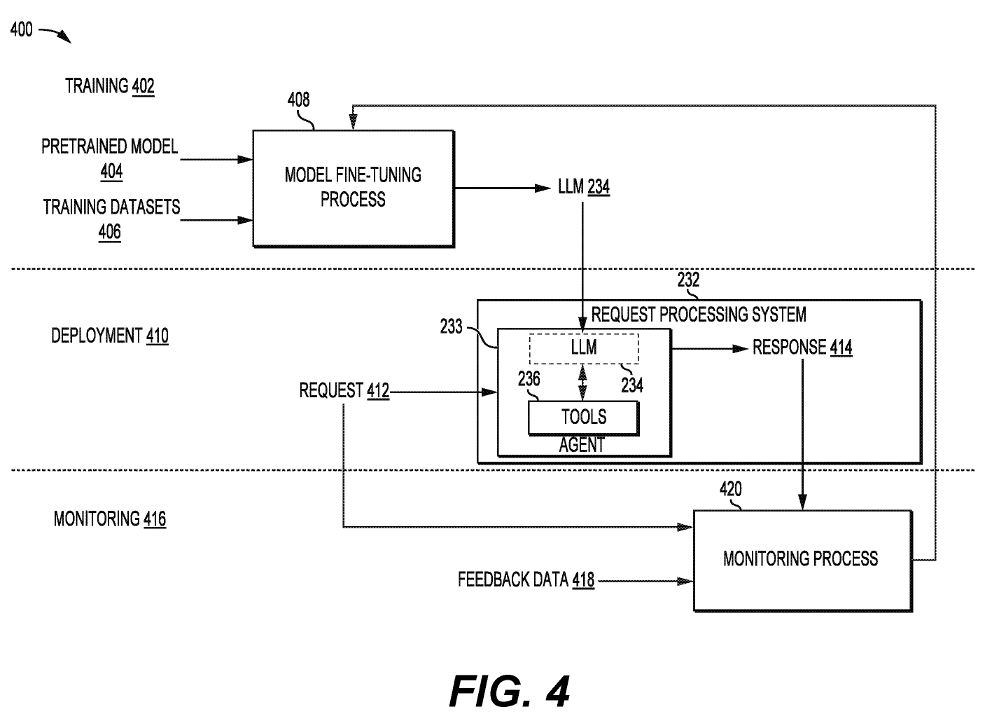

At a high level, and as described in more detail with reference to FIG. 3, LLM 234 may at least be aware of tools 236, and, based on the text request received as input, LLM 234 may generate code to indicate one of tools 236 is to be invoked. In some examples, a listing of tools 236 may be provided to LLM 234 as part of a system prompt along with the text request. In other examples, and as described in in more detail with reference to FIG. 4, LLM 234 may be trained or fine-tuned to predict a tool to call from among tools 236. The code indicating the tool to be invoked may be included in an initial output of LLM 234. Agent 233 may parse the initial output, recognize that a tool call is required, and invoke the tool on behalf of LLM 234. An output from the tool (e.g., a result) will then be sent back to LLM 234 via agent 233. Agent 233 may augment a dynamic or updatable portion of the prompt including the text request (e.g., the general prompt) based on the result, and LLM 234 may process the augmented prompt to generate a response as final output of LLM 234.

Example types of tools 236 may include tools associated with functions for controlling features of physical devices, such as computing system 104. When called, these functions may return, as a result, commands having a syntax specific to computing system 104. For example, the commands may include a specific structure and format understood and used by computing system 104 to perform tasks. As a result, the commands are readable and executable by computing system 104 upon receipt.

One illustrative example includes a brightness function for adjusting the amount of light emitted by lighting device 112 via control signals that are generated by computing system 104 and transmitted to lighting device 112. Other operating parameters of lighting device 112 or imaging device 110 may be similarly controlled or adjusted. Another illustrative example includes an image enhancement function for turning on or off an image enhancement feature of computing system 104 that, when turned on, processes images captured by and received from imaging device 110 using an image enhancement algorithm to generate and display enhanced images. Further illustrative examples include an image capture function or video record function for recording and storing an image or video, respectively, captured by and received from imaging device 110. The above-described functions are illustrative and non-exhaustive, a tool and corresponding function of the tool may be defined for any operations or tasks performable by computing system 104 to control aspects of computing system 104 or other devices communicatively coupled to computing system 104.

Other example types of tools 236 may be associated with functions to enable interactions with additional systems 240. Additional systems 240 may be internal or external systems that are configured to perform a specific task or action, and specifically a task or action that may obtain useful information for responding to the request. A video retrieval system 242 may be one of additional systems 240 that request processing system 232 can interact with via one of tools 236. For example, the tool may be invoked when the request includes an issued command to provide a medical procedure-related video or other similar medical content.

At step 218, method 210 may include providing the response to computing system 104. Once LLM 234 has generated the response, the response may be transmitted in text format (e.g., as a text response) back to serverless compute system 226. Serverless compute system 226 may then upload a text file including the response onto data object storage system 224. The text file including the response may be stored on data object storage system 224 in association with the audio file including the voice request.

In some aspects, serverless compute system 226 may perform other actions using data that is stored by data object storage system 224, including request and response data. For example, serverless compute system 226 may perform data analytics to identify a number of uses of the assistant feature, types or categories of requests submitted via this feature, or operator satisfaction with responses provided to the requests, among other similar metrics.

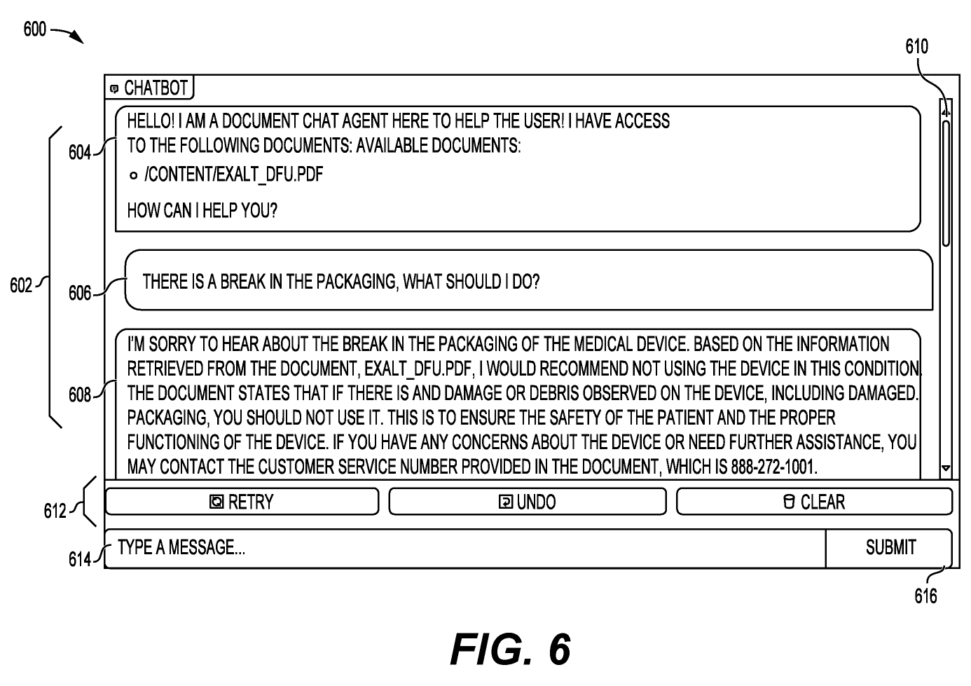

Returning to FIG. 2A, at step 208, method 200 may include receiving, at computing system 104, a response to the voice request. In some examples, computing system 104 may query for the text file including the response that is stored in data object storage system 224 via IoT platform 222 and download the text file. After the download, computing system 104 may display the response in the text format via a user interface, such as a chatbot application user interface, presented on a display of computing system 104 (e.g., one of display device(s) 106) for the operator’s review. An example user interface is shown and described with reference to FIG. 6. In some examples, in addition to, or alternatively from, the text display, computing system 104 may be configured to present the response in an audio format. For example, the text file may be converted to an audio filed using a text-to-speech model executing locally on computing system 104 or one of edge device(s) 130, or remotely by one of server- side system(s) 140 along the request processing pipeline 220.

In some examples, dependent on a type of the voice request received, additional actions or tasks may be performed by computing system 104 based on the response. For example, and as described in more detail with reference to FIG. 3, if the voice request is to control an operating parameter of a component of medical system 100, the response may include a command that is read and executed by computing system 104 to cause that operating parameter to be controlled accordingly (e.g., through generation and transmission of control signals by computing system 104). As another example, and as described in more detail with reference to , FIGS. 5A5-5C,if the voice request is to play back a video showing an aspect of a medical procedure, the response may include a playback command, along with video data and a timestamp to cause the video to be played back at a corresponding time in the video that is relevant to (e.g., shows) the requested aspect of the medical procedure.

Method 200 and method 210 described above are provided merely as examples, and may include additional, fewer, different, or differently arranged steps than depicted in FIGS. 2A and 2B.

FIGS. 2A-2C describe use of request processing pipeline 220 to process a voice request received at computing system 104. However, in other aspects, a text request may be received at computing system 104 and similarly processed via at least a portion of request processing pipeline 220. For example, text may be provided as input to computing system 104 via a touch user interface of computing system 104 or a peripheral input device communicatively coupled to computing system 104 (e.g., a keyboard). In some examples, the text may be input via a text field of a chatbot application user interface of the chatbot application running on computing system 104. Additionally or alternatively, one or more user interfaces of computing system 104 may include a dedicated graphical control element associated with a particular type of command displayed thereon that is selectable by an operator to provide that particular type of command as a text request. One example dedicated graphical control element may be a transcribe medical procedure element to request a transcription of an audio recording of the medical procedure. If such as control element is selected, computing system 104 may automatically generate the request in a text format for transmitting server-side system(s) 140 of request processing pipeline 220.

The text request is transmitted in a text data format (e.g., as a text file) when uploaded by computing system 104 via IoT platform 222 and stored on data object storage system 224. Therefore, when detected, serverless compute system 226 may invoke LLM 234 associated with request processing system 232, and pass the text request to LLM 234 for processing. In other words, serverless compute system can bypass interacting with transcription system 228 within request processing pipeline 220.

In further aspects, portions of request processing pipeline 220 may be configured to perform other tasks or processes, such as a transcription process. For example, a transcription feature of computing system 104 may be turned on or otherwise enabled (e.g., through selection of a graphical control element via a user interface of computing system 104 or through a text or voice request processed as described above). In such examples, the transcription process may leverage request processing pipeline 220, except for request processing system 232. For example, the transcription process may include steps similar to steps 204-206 of method 200 and steps 212-214 of method 210, except that, instead of a recording of a voice request being captured and transcribed, a medical procedure is being continuously recorded and transcribed.

In other examples, if the transcription feature is enabled, and video data captured by imaging device 110 is being continuously recorded (in addition to or instead of audio data), transcription system 228 may leverage one or more VLMs configured to process the video data to generate text that describes what is occurring throughout the video. To help train the VLM, or retrain the VLM to improve an accuracy thereof, the text generated and output by the VLM for a video recording may be compared to a transcript generated from a simultaneously recorded audio recording, as described above.

Once the audio content (or video content) of the medical procedure is transcribed by transcription system 228 at step 214, the transcript is passed back to serverless compute system 226. Serverless compute system 226 then uploads a transcription file containing the transcribed text onto data object storage system 224. Computing system 104 may query for and download the transcription file from data object storage system 224 via IoT platform 222. Once downloaded, computing system 104 indicates availability of the transcription file.

In some examples, the operator may then initiate an upload, or the transcription file may otherwise be automatically uploaded, to a remote storage system, such as a PACS system for storage in association with images or videos captured throughout the medical procedure. In some examples, the transcript, the images, and videos may each include timestamps from the procedure that are stored as metadata of each the respective document type. Therefore, using the respective timestamps, a correspondence between a portion of the transcript describing an anomaly in an anatomical structure and an image of the anomaly captured while the operator was describing the anomaly may be identified, and stored in combination with one another.

FIGS. 2A-2C describe one example aspect of request processing that leverages an at least partially remote request processing pipeline 220 formed by server-side system(s) 140. In other examples, the request processing may be performed entirely by computing system 104, a combination of computing system 104 and edge device(s) 130, or a combination of computing system 104, edge device(s) 130, and server-side system(s) 140. When the request processing is performed by computing system 104 or edge device(s) 130, the above-described models and processes of at least transcription system 228 and request processing system 232 may be executed by computing system 104 or edge device(s) 130 to perform steps 214 and 216, respectively.

Further, FIGS. 2A-2C describe transcription system 228 and request processing system 232 as separate systems of request processing pipeline 220. In other examples, functions of transcription system 228 may be integrated with request processing system 232. For example, request processing system 232 may receive, via serverless compute system 226, the recording (e.g., in the audio data format). STT model 230 may then be executed by request processing system 232 to transcribe the audio content of the recording to text for further processing, via the agent framework of request processing system 232, to generate the response.

FIG. 3 depicts an example method 300 for generating a response to a voice request. For example, one or more steps of method 300 may be implemented to perform step 216 of method 210, described above with reference to FIG. 2B. One or more steps of method 300 may be performed by request processing system 232, and specifically agent 233 or LLM 234.

At step 302, method 300 may include receiving a prompt including the text of the voice request (e.g., the text generated by transcription system 228). The text of the voice request may be in a form of a message that is concatenated and formatted (e.g., using templates) for inclusion within the prompt such that the text is understandable by LLM 234. At least one or more portions of the prompt, such as a general prompt including the text of the voice request, may be dynamic and updateable, as described below. In some examples, other portions of the prompt, such as the system prompt, may be set or static. In other examples, the system prompt may also be dynamic and updateable based on checklists, for example. The system prompt may define how the LLM 234 should behave to respond to the prompt. In some examples, the system prompt may also provide information about (e.g., a listing of) tools 236 available to LLM 234. The information for each tool may include a textual description of what a corresponding function of the tool does, the function or callable, expected inputs (e.g., arguments) to the tool, and expected outputs (e.g., results) of the tool. In other examples, and as described in more detail with reference to FIG. 4, LLM 234 may be fine-tuned to learn tool or function calling.

At step 304, method 300 may include identifying a tool (e.g., from tools 236) to invoke based on the prompt. One or more tools 236 may be invoked when they are needed to retrieve data, perform specific actions or tasks, and/or interact with additional systems 240 in order to respond to or otherwise fulfill the voice request. The determination may be based on the text of the voice request. For example, the text may be analyzed to determine an intent or meaning (e.g., what does the operator need). Based on the intent or meaning, a tool, from tools 236, may be identified as a tool needed to fulfill the request.

In some examples, the tool may be identified using the listing of available tools 236 included in the prompt in view of the text of the voice request. In other examples, when LLM 234 is fine-tuned to learn tool or function calling, LLM 234 may, based on the text, identify or predict a token corresponding to the tool to call. For example, the text may be analyzed to identify any words or phrases associated with the token corresponding to the tool (e.g., a token learned by LLM 234), as described in more detail with reference to FIG. 4.

To provide an illustrative example, the voice request may be a query of "How do you use a component of medical device 102?", and the identified tool may be a RAG-associated tool for accessing the DFU data store. To provide another illustrative example, the voice request may be an issued command to "Increase brightness of lighting device 112," and the identified tool may correspond to a brightness function. To provide a further illustrative example, the voice request may be an issued command to "Playback a video of a cannulation of a papilla," and the identified tool may correspond to a video retrieval function.

In other examples, if no tool is able to be identified at step 304, a default response may be generated and method 300 may end. For example, if the request includes a question related to a topic outside of guardrails defined in the system prompt, a response indicating that the assistant feature does not know the answer may be generated. As another example, if the request includes a task that none of tools 236 are capable of performing, a response indicating that the assistant feature does not have the capability to perform the requested task may be generated. To provide an illustrative example, none of tools 236 may be defined to email an image captured by medical device 102.

At step 306, method 300 may include calling the tool with inputs from the prompt. When the tool is determined to be invoked, LLM 234 may generate code to indicate the tool to be invoked. The code may be included in an initial output of LLM 234 (e.g., output responsive to the prompt received at step 302 forming an initial response based on the text of the voice request). Agent 233 may parse the initial output of LLM 234, recognize that a tool call is required based on the code included, and invoke the tool on behalf of LLM 234 using the code. The code may include the inputs to the tool from the prompt needed by the tool to execute the corresponding function for the tool.

At step 308, method 300 may include receiving, from the tool, a result generated from an execution of the function corresponding to the tool using the inputs. The result may be the output generated by the tool in response to executing the function using the inputs.

Continuing with the example where the voice request may be a query of "How do you use a component of medical device 102?", and the RAG-associated tool for accessing the DFU data store is called, the result may include relevant or contextual information associated with the component of the particular model of medical device 102 retrieved from the DFU data store. Continuing with the example where the voice request may indicate to "Increase brightness of lighting device 112," and the brightness function is called, the result may include a corresponding command having a syntax specific to computing system 104. The corresponding command may include a specific structure and format understood and used by computing system 104 to perform tasks, and specifically to increase the brightness of the lighting device 112.

At step 310, method 300 may include generating the response to the voice request based on the text and the result. In some examples, the result may be added, by agent 233, to the dynamic or updateable portion of the prompt including the text request (e.g., the general prompt) to generate an augmented prompt. LLM 234 may then process the augmented prompt to generate the response.

Continuing with the example where the voice request may be a query of "How do you use a component of medical device 102?", and the result may include the relevant or contextual information associated with the component of the particular model of medical device 102, the response may include or may be generated further based on the relevant or contextual information. Continuing with the example where the voice request may indicate to "Increase brightness of lighting device 112," and the result includes the corresponding command having a syntax specific to computing system 104, the response may include the corresponding command. Resultantly, once the response is provided to the computing system 104, computing system 104 may be configured to read and execute the command to increase the brightness of lighting device 112 (e.g., by generating and transmitting control signals from computing system 104 to lighting device 112).

Identification and calling of one tool responsive to the prompt is described herein for brevity and clarity. However, in other examples, multiple tools, from tools 236, may be identified and called in response to a single prompt. In such examples, the results from each of the multiple tools may be similarly obtained and used to determine or generate the response. Additionally, while generating a response to a voice request is described, method 300 may be performed to similarly generate a response to a text request.

Method 300 described above is provided merely as an example, and may include additional, fewer, different, or differently arranged steps than depicted in FIG. 3.

FIG. 4 is a block diagram showing an example process 400 for training, deploying, and monitoring a machine learning model, such as LLM 234. In some examples, a system or device configured to generate or train LLM 234 may be a same system or device configured to deploy or use LLM 234. In other examples, the system or device configured to generate and/or train LLM 234 may, upon completion of training, provide LLM 234 to a different system or device for storage, and subsequent deployment. As shown in FIG. 2C, LLM 234 may be deployed by request processing system 232 that is remotely located (e.g., in the cloud). In other examples, LLM 234 may be deployed by a local machine, such as one of edge device(s) 130 or computing system 104.

Process 400 may include a training phase 402, a deployment phase 410, and a monitoring phase 416. In training phase 402, a pretrained model 404 may be fine-tuned with a plurality of training datasets 406 as part of a model fine-tuning process 408 to build LLM 234. For example, model fine-tuning process 408 may apply low rank adaption (Lora) techniques to enable LLM 234 to learn how to call tools 236 as part of request processing. LLM 234 may also consist of a base model with selectable LoRA adapters that have been trained for different purposes.

The pretrained model 404 may be an off-the-shelf or foundational LLM received from internal or external resources. An example training dataset 406 may include text of a request for which a tool is to be called, and a ground truth including a response to the request and a unique token associated with the tool to be called to obtain information for generating the response. To provide an illustrative example, within an example training dataset 406, the text of the request may be "Turn lighting device brightness up". The corresponding ground truth includes a response having a command with a syntax specific to computing system 104 to perform the action or task of increasing a brightness of lighting device 112, and a unique token included in the text and associated with a tool that is called to obtain the command (e.g., a brightness function). For example, the unique token may be "ADJ_BRI". Training datasets 406 may include internally collected requests and responses or request and response pairs obtained from a third party or public database.

Generally, a model, such as pretrained model 404, includes a set of variables, e.g., nodes, neurons, filters, etc., that are tuned, e.g., weighted or biased, to different values via the application of training datasets 406. Specifically, via application of the LoRA techniques, a layer of weights or trainable parameters is added to pretrained model 404. The weights or parameters are then trained, and resulting LLM 234 learns a token associated with each of tools 236. For example, as part of the learning process, tokens may be added as training datasets 406 are processed. Adding tokens essentially adds vocabulary or words understood by LLM 234. Continuing with the above example, LLM 234 learns the unique token (e.g., "ADJ_BRI") associated with the brightness function, such that when text of a future received request includes "brightness" and “adjust”, or other similar terms or phrases, the brightness function is called. To provide another illustrative example, LLM 234 may learn a unique token (e.g., "REC_VID") associated with a video retrieval function, such that when text of a future received request includes "video" and “receive”, or other similar terms or phrases, the video retrieval function is called to initiate interactions with video retrieval system 242, as described in detail with reference to FIG. 5A-5C below.

In some examples, model fine-tuning process 408 employs supervised, unsupervised, semi-supervised, or reinforcement learning processes to fine-tune pretrained model 404 (e.g., to result in LLM 234). In some aspects, a portion of training datasets 406 are withheld during training or used to validate LLM 234.

When supervised learning processes are employed, the ground truth included in training datasets 406 facilitates the learning. For example, training proceeds by feeding a request from a training dataset 406 (e.g., a sample) into pretrained model 404, where pretrained model 404 has variables set at initialized values. Pretrained model 404 outputs a predicted response that is obtained by identifying and calling a tool from tools 236 (e.g., via identifying an associated token) based on text of the request. The output is compared with the ground truth to determine an error, which is then backpropagated through the model to adjust the values of the variables. This process is repeated for a plurality of samples at least until a determined loss or error reaches an acceptable threshold to build LLM 234. In some examples, a portion of training datasets 406 are withheld and used to further validate or test LLM 234.

Once fine-tuned, LLM 234 may be stored and subsequently deployed. During deployment phase 410, a request 412 is received by LLM 234 as part of a prompt, as described in detail with reference to FIG. 3. Based on text of request 412, LLM 234 may identify a token, and generate code to indicate that one of tools 236 associated with the token is to be invoked. The code may be included in an initial output of LLM 234. Agent 233 may parse the initial output of LLM 234, recognize that a tool call is required based on the code included, and invoke the tool on behalf of LLM 234 using the code. The code may include the inputs to the tool from the prompt needed by the tool to execute the corresponding function for the tool. Agent 233 may receive a result from the tool, and add the result to a dynamic or updatable portion of the prompt (e.g., a general prompt including request 412) to generate an augmented prompt. LLM 234 may then process the augmented prompt to generate and output a response 414.

During monitoring phase 416, feedback data 418 related to response 414 may be collected. Feedback data 418 may include an indication of an accuracy of response 414. In some examples, feedback data 418 may be based on human expert feedback (e.g., model developers and/or operators). In other examples, feedback data 418 may be based on a model-based analysis (e.g., using LLM-as-a-judge). In further examples, while an LLM-as-a judge may be implemented as an initial analyzer of response 414, the LLM analysis may be provided to the human expert as a data point for use in assessing the response 414 and ultimately the human expert may generate feedback data 418. Feedback data 418, along with request 412 and response 414, may be input into a monitoring process 420 for analysis to determine an accuracy of response 414. In some examples, based on the analysis, process 400 returns to training phase 402, where at model fine-tuning process 408 the values of one or more variables of LLM 234 are adjusted to re-train or further fine-tune LLM 234. For example, a new training dataset 406 may be generated based on request 412 and feedback data 418 (e.g., feedback data 418 may include an accurate response 414 to request 412), and fed into LLM 234 to cause the adjusting of the variables.

In addition to, or alternatively from, retraining LLM 234, feedback data 418 may be used to flag edge cases or unpredicted results. Further, feedback data 418 may be used to improvise the prompt dynamically.

Process 400 described above is provided merely as an example, and can include additional, fewer, different, or differently arranged aspects than depicted in FIG. 4.

FIGS. 5A-5C depict example processes 500, 520 and a system flow 550 for establishing one or more search data stores (e.g., one or more of data stores 238) for subsequent use in processing a request associated with retrieving video or other medical content.

Specifically, FIG. 5A depicts example process 500 for establishing the search data stores. In some examples, process 500 may be performed by, and thus the search data stores may be established by video retrieval system 242. In other examples, process 500 may be performed by a third party system separate from video retrieval system 242, and the search data stores established by the third party system may be stored at a location accessible by video retrieval system 242 to enable subsequent use or querying thereof. Process 500 may represent an establishment phase 551, as shown in a top portion (e.g., above the horizontal broken line) of system flow 550 of FIG. 5C. Process 500 may include at least an establishment of a video search data store 566, as described with reference to steps 502-512. In some examples, in addition to video search data store 566, an optional medical search data store 568 may be established, as described with reference to step 514.

Referring concurrently to FIG. 5A and establishment phase 551 of FIG. 5C, at step 502, process 500 may include receiving video data 554 of a video associated with a medical procedure. The video may be one of a plurality of videos received, as part of a data collection process 552, from one or a combination of internal and external resources. For example, the videos may be received from medical facility archives, medical training platforms, or other similar trusted medical sources. The videos can include training videos or videos recorded during medical procedures at a same or different facility by one or more operators.

Data pre-processing 558 may then be performed on video data 554. For example, at step 504, process 500 may include generating a video transcript from video data 554. In some aspects, the video transcript may be generated based on audio data (e.g., is a text transcript representing spoken words or other sounds captured in the video data 554). To generate the video transcript, the audio data may initially be extracted from video data 554 and enhanced. The enhanced audio data may then be transcribed to generate the video transcript. The enhanced audio data may be transcribed using transcription system 228 or a separate system having a model similar to STT model 230 deployed by transcription system 228. In some examples, the video transcript may be generated in a JSON format.

In other aspects, the video transcript may be generated from video data 554 by applying a vision language model (VLM). In such examples, video data 554 of the video may be input to the VLM. The VLM may process video data 554 to generate, as output, text describing what is occurring throughout the video based on visual information extracted from video data 554.

Additionally, as part of data pre-processing 558, at step 506, process 500 may include extracting metadata associated with video data 554. One or more different types of metadata may be extracted. Dependent on the metadata type, the metadata may be extracted from video data 554 or the video transcript generated from video data 554 at step 504. In some examples, rather than using the VLM to generate the video transcript, the VLM may be used to extract, as one type of metadata, visual features from the video data 554 to enhance the audio data-based video transcript.

Another type of metadata that may be extracted includes a plurality of timestamps from video data 554. In some examples, the video transcript may be divided into a plurality of portions or chunks of text comprising the video transcript. The extracted timestamps from video data 554 may correspond to the portions of text representing speech or other sounds occurring during the video at that given timing.

Yet another type of metadata that may be extracted includes speaker information. For example, the extracted and enhanced audio data may be further processed or analyzed to identify one or more speakers based on a spectrum analysis of speech signals. For example, speech signals captured from different speakers may include variations in spectral features that enables speech from one speaker to be distinguished from another speaker. By performing such analysis, a core or authentic speaker (e.g., a physician) may be identified. In some examples, an indication of a speaker (e.g., core or otherwise), if any, identified at each of the extracted timestamps may be provided as metadata to enable association with respective segments of the video and corresponding portions of text from the video transcript based on the timestamps. In further examples, a particular identity of the speaker or a source of the video (e.g., an affiliated facility), among other similar data, signaling trust or reliability in the video, may also be extracted for subsequent association.

A further type of metadata that may be extracted includes medical terms. For example, the portions of text comprising the video transcript may be evaluated to determine a presence of one or more medical terms from a pre-defined listing of medical terms. Any medical terms determined to be present within a given portion may be extracted and associated with the portion of text.

At step 508, process 500 may include storing video data 554, the video transcript, and the metadata in a video data store 560. At least the video transcript and the metadata may be stored in a structured format. For example, each of the portions of text comprising the video transcript may be stored in association with corresponding timestamps, speaker information, medical terms, or other metadata.

In some examples, the stored metadata may serve as tags or labels for each segment of video data 554 and corresponding portion of video transcript. Additionally, or alternatively, the metadata may also be used as filtering criteria. For example, and as described in more detail below, only a subset of the portions of video transcript may be identified for transformation based on the metadata. As one illustrative example, any portions of video transcript with metadata indicating no speaker or a speaker other than the core or authentic speaker may be discarded such that they are not transformed. As another illustrative example, only portions of video transcript with metadata indicating inclusion of one or more medical terms may be transformed.

At step 510, process 500 may include transforming one or more of the plurality of portions of the video transcript to generate one or more transformed portions of the video transcript (e.g., using a data transformation process 564). In some examples, the transformed portions include each of the plurality of portions of the video transcript. In other examples, the transformed portions include only a subset of the plurality of portions of the video transcript based on filtering criteria applied, as described above. A type of data transformation process 564 performed to generate the transformed portions of the video transcript may be based on a type of search data store to be established or built. For example, the portions of the video transcript may be transformed into a format compatible for storage in the search data store.

As one example, video search data store 566 may be a vector database that is established to facilitate similarity searching. A vector database is configured to store the portions of text as vector representations of the portions of text (e.g., as vector embeddings) in a vector space. A vector embedding for a text portion may be a mathematical or numerical representation of the text portion generated based on one or more of the attributes of the text portion. In such examples, data transformation process 564 may include conversion of the one or more portions of video transcripts into one or more corresponding vector embeddings.

To convert a portion of video transcript, text of the portion may be divided into a plurality of tokens using sentence, word, sub-word, and/or character-based tokenization, among other tokenization techniques known in the art. A vector embedding for the portion may be generated based on the tokens, where the vector embedding mathematically or numerically represents the meaning of and/or relationship among the tokens. The vector embedding may then be stored in the vector space.

As another example, video search data store 566 may be a graph database. An example graph database may represent video transcripts of videos as a hierarchy of nodes and relationships between the nodes as edges in the form of graph embeddings. To transform a video transcript for storage in the graph database, the text of the video transcript may be chunked (e.g., divided into chunks) and associated metadata of the video transcript may be analyzed in view of the structure of the nodes and edges within the graph database to generate a graph embedding for each chunk (e.g., to transform nodes and edges into a vector representation).

Non-limiting and non-exhaustive examples of nodes may include nodes associated with a chunk, a medical entity, a topic, a decision point, a timestamp, a speaker, and a multimodal asset. The chunk node may represent a smaller segment or portion (e.g., a chunk) of a video transcript of a video with an associated time and source. The chunk node may be sequentially connected to other chuck nodes representing chunks that either precede or follow the present chunk (e.g., via NEXT edge). In some examples, the timestamp nodes may facilitate linking or connecting of sequences. Additionally, if the chunk is associated with or encompasses a topic representing a higher-level chunk group, the chunk node may be connected to the topic node (e.. g, via PART_OF-TOPIC edge).

Further, if the chunk mentions an entity represented by a medical entity node (e.g., a symptom, a procedure, a parameter, a device), the chunk node may be connected to the medical entity node (e.g., via MENTIONS edge). Similarly, if an entity is mentioned by a speaker represented by a speaker node (e.g., one of the operator, an assistant, or device in environment), the speaker node may be connected to the respective medical entity node (e.g., via a MENTION_BY edge). Medical entity nodes having a link or relationship to one another may also be connected to one another (e.g., via RELATED_TO edge). Also, if a symptom represented by a medical entity node present in the chunk leads to a clinical decision (or branching) represented by an decision point node, the medical entity node may be connected to the decision point node (e.g., via a TRIGGERED_BY edge). Similarly, if a parameter represented by a medical entity node present in the chunk leads to an action-based decision, the medical entity node may be connected to the decision point node (e.g., via a DECISION _ON edge).

In some examples, a chunk may be linked or otherwise associated with media, including one or more a combination of a video, audio, or image frame or segment. In such examples, nodes may include one or more multimodal asset nodes representing or otherwise pointing to locations of the media (e.g., via resource locators). The chunk node representing the chunk may then be connected to the multimodal asset node(s) (e.g. via a HAS_ASSET edge).

At step 512, process 500 may include storing the one or more transformed portions of the video transcript in video search data store 566. To establish video search data store 566, steps 502 through 512 may be repeated for each video of the plurality of video received as part of data collection process 552. Resultantly, video search data store 566 may include one or more transformed portions of each of a plurality of video transcripts corresponding to the plurality of videos. Additionally, as new video data 554 is collected (e.g., at predefined intervals), steps 502 through 512 may be repeated to periodically update video search data store 566. In some examples, process 500 may be performed on new video data 554 in batches (e.g., when a threshold amount of new video data 554 has been collected).

In some aspects, process 500 may include an optional step 514 for establishing optional medical search data store 568 by receiving and processing medical data 556. For example, as part of data collection process 552, optional medical data 556 may be received from one or more internal or external resources. Optional medical data 556 may be associated with medical procedures. In some examples, optional medical data 556 may be web-based content scraped from trusted medical websites for relevant case studies, research articles, or procedural guidelines. As part of data pre-processing 558, optional medical data 556 may be parsed and cleaned to generate structured text for storage in optional medical data store 562. The structured text of medical data 556 may then be transformed using data transformation process 564, similar to text portions of the video transcript, to index the structured text within optional medical search data store 568 to facilitate searching.

Process 500 described above is provided merely as an example, and may include additional, fewer, different, or differently arranged steps than depicted in FIG. 5A.

FIG. 5B depicts an example process 520 for processing a request using one or more search data stores established via process 500 of FIG. 5A, including at least video search data store 566. For example, process 520 may represent a deployment phase 570, as shown in a bottom portion (e.g., below the horizontal broken line) of system flow of FIG. 5C. One or more steps of process 520 may be performed by video retrieval system 242. As depicted in FIG. 2C, video retrieval system 242 may be a remote system that is one of additional systems 240 configured to interact with request processing system 232 as part of the request processing pipeline 220. However, in other examples, video retrieval system 242 may be executed on a local machine, such as one of edge device(s) 130 or on computing system 104.

Referring concurrently to FIG. 5B and deployment phase 570 of FIG. 5C, at step 522, process 520 may include receiving text of a request. In some examples, the request may be originally provided as a voice request, such as the voice request captured within the recording at step 204 of method 200. In such examples, the text of the request may be the text generated from the transcription of audio content of the recording at step 214 of method 210. In other examples, the request may be originally provided as a text request.

In some aspects, video retrieval system 242 may be configured to interact with request processing system 232 via one of tools 236. In such examples, the text of the request may be received as part of a tool call at step 306 of method 300. To provide an illustrative example, the text may include “Playback a video of a cannulation of a papilla.” In some examples, the text may further include a specific source from which the video is requested (e.g., archives of the current facility) or identify a specific operator (e.g., speaker) of the video, such as a mentor or a well-known expert of the procedure in the field. Based on the unique token (e.g., "video") associated with the video retrieval function learned by LLM 234, upon receiving an original prompt including the text, LLM 234 may generate code to indicate a tool providing the video retrieval function is to be called for inclusion in initial output of LLM 234. Agent 233 may recognize and call video retrieval system 242 on behalf of LLM 234. As part of the call, the text of the request may be provided to video retrieval system 242.

In other aspects, video retrieval system 242 may be configured to receive the request directly from computing system 104, one of edge device(s) 130, or another one of server-side system(s) 140 in request processing pipeline 220 (e.g., from transcription system 228).

At step 524, process 520 may include transforming the text via data transformation process 572 to generate transformed text. Data transformation process 572 may transform the text in a similar manner (e.g., to a similar structure or format) as data transformation process 564 used to establish video search data store 566 as described. For example, when video search data store 566 is a vector database, the transformed text may include a request vector embedding. Specifically, data transformation process 572 may include dividing (or chunking) the text into a plurality of tokens using sentence, word, sub-word, and/or character-based tokenization, among other tokenization techniques known in the art. The request vector embedding may then be generated based on the chunked tokens, where the request vector embedding mathematically or numerically represents the meaning of and/or relationship among the tokens. As another example, when video search data store 566 is a graph database, the transformed text may include a request graph embedding to represent nodes and edges in a vector format.

At step 526, process 520 may include identifying, from video search data store 566, a transformed portion of video transcript based on the transformed text. For example, as part of a search process 574, video retrieval system 242 may query video search data store 566 using the transformed text to identify a relevant transformed portion of video transcript.

When video search data store 566 is a vector database and the transformed text is a request vector embedding, a vector embedding in close proximity to (e.g., within a predefined distance of) the request vector embedding in the vector space may be identified. The identified vector embedding may also be referred to as a similar or matching vector embedding. The close proximity indicates a similarity in text that the transformed portion of video transcript is representing with the text included in the request, and thus a relevancy or responsiveness of the transformed portion of video transcript (and corresponding video segment) to the request. Continuing with the above illustrative example where the text of the request may include “Playback a video of a cannulation of a papilla”, the transformed portion of video transcript may include the terms cannulation and papilla.

As another example, when video search data store 566 is a graph database, and the transformed text is a request graph embedding, one or more similar graph embeddings to the request graph embedding may be identified within the graph database.

At step 528, process 520 may include retrieving, from video data store 560, a portion of video transcript corresponding to the transformed portion of video transcript, a timestamp associated with the portion of video transcript, and video data associated with the video transcript to generate a result. For example, as part of result retrieval process 578, video retrieval system 242 may query video data store 560 for the portion of video transcript corresponding to the transformed portion of video transcript. The timestamp and video data may be stored in association with, and retrieved in addition to, the portion of the video transcript. The portion of the video transcript, the time stamp, and the video data may be included in the result.

For brevity and clarity, identifying one transformed portion of video transcript has been described herein. However, in other examples, a plurality transformed portions of video transcript may be identified as relevant to the transformed text. In such examples, a portion of video transcript corresponding to each of the transformed portion of video transcripts may be retrieved, along with the timestamp associated with each portion of video transcript, and video data associated with each video transcript for use in generating the result. Further, in examples where the text of the request may further include a specific source from which the video is requested (e.g., archives of the current facility) or identify a specific operator (e.g., speaker) of the video, the speaker identity or source of the video data may be used to query video data store 560. As a result, only a subset of one or more of the transformed portions of video transcripts identified as relevant to the transformed text that have matching metadata to the speaker identity or source may be returned for use in generating the result.

At step 530, process 520 may include providing the result to request processing system 232 for use in generating a response to the request. For example, agent 233 may receive the result, and add the result to a dynamic or updateable portion of the original prompt received by LLM 234 to generate an augmented prompt. LLM 234 may process the augmented prompt to generate the response. For example, the response may include at least the video data of the video, and a command with appropriate syntax to cause the video data to be played back at computing system 104 at a time corresponding to the timestamp associated with the portion of the video transcript (e.g., the relevant portion). Continuing with the above-illustrated example, the video data may include an entirety of an Endoscopic Retrograde Cholangiopancreatography (ERCP) procedure; however, the video data will be played starting at the time indicated by the timestamp associated with the portion of the video transcript that includes the terms cannulation and papilla. As a result, the segment of the video data showing the cannulation of the papilla is played at computing system 104. In some examples, the response may also include the portion of the video transcript, and the portion of the video transcript may be displayed in conjunction with the playing of the video segment.

In some aspects, process 520 may include an optional step 532 for identifying, from medical search data store 568, a transformed portion of medical data based on the transformed text to retrieve and provide, as part of the result, a portion of medical data 556 corresponding to the transformed portion of medical data. For example, as part of search process 574, video retrieval system 242 may query optional medical search data store 568 using the transformed text to identify a relevant transformed portion of medical data. Then, as part of result retrieval process 578, video retrieval system 242 may query optional medical data store 562 for the portion of medical data 556 corresponding to the transformed portion of medical data.

In some examples, a first relevancy of the transformed portion of video transcript identified from video search data store 566 to the request may be quantified and compared to a second relevancy of the transformed portion of medical data 556 identified from optional medical search data store 568 to the text of the request (e.g., via a relevancy comparison process 576). In examples where vector-based searching is used, relevancy may be quantified using cosine similarity. For example, a cosine similarity between the request vector embedding and the vector embedding representing the portion of the video transcript or the portion of medical data may be determined. Cosine similarity may be measured as a scalar (e.g., from a dot product of 2 vectors) and return a value from -1 to 1, where a value of 1 indicates a highest similarity with vectors pointing in a same direction. In examples where graph-based search is used, relevancy between the request graph embedding and the graph embedding representing the portion of the video transcript or the portion of medical data may be quantified using one or a combination of techniques. One example technique may include an approximate nearest neighbor (AAN) search using a graph-based algorithm, such as hierarchical navigable small world (HSNW). Another example technique may include a vector plus graph traversal to identify previous and next nodes, as well as to identify conceptual relationships, symptoms, or devices. Yet another example technique may include keyword matching with weight boosting for relevant keywords. A further example technique may include an ontology-based approach, such as entity or concept overlap where relevancy is based on a shared concept between the request and a node. Based on the comparison, one or both of the portion of video transcript or the portion of medical data 556 may be included as part of the result generated. In some examples, inclusion as part of the result may be based on whether the quantified relevancy meets one or more thresholds.

Process 520 described above is provided merely as an example, and may include additional, fewer, different, or differently arranged steps than depicted in FIG. 5B.

FIG. 6 depicts an example user interface 600. User interface 600 may be displayed on one of display device(s) 106, such as the display of computing system 104. In some examples, user interface 600 may be a user interface of the chatbot application that is executing locally or via a web browser on computing system 104.

User interface 600 may display a conversation 602 between a chatbot and an operator. Conversation 602 may include an introduction 604 from the chatbot indicating capabilities of the chatbot to process requests from the operator. Additionally, introduction 604 may indicate any knowledge databases or documents available to facilitate the request processing. For example, based on a type of medical device 102 connected to computing system 104, introduction 604 may indicate available access to a DFU document for the medical device type. Introduction 604 may further prompt the operator to submit a request (e.g., “How can I help you?”).

Conversation 602 may also include a request 606 received from the operator (e.g., “There is a break in the packaging, what should I do?”). In some examples, request 606 may initially be received as a voice request that is processed via both transcription system 228 to generate text and request processing system 232 to generate a response 608 based on the text, as described above in detail with reference to FIGS. 2A-2C. In other examples, request 606 may be received in a text format. For example, text may be input via a text field 614, and submitted upon a selection of a “submit” graphical element 616 displayed on user interface 600. In such examples, transcription system 228 may be bypassed, and the text may be processed by request processing system to generate response 608. Response 608 may then be displayed within conversation 602.

Depending on a length of conversation 602 and a display size, user interface 600 may further display a scroll bar 610 associated with conversation 602 to enable the operator to scroll through or navigate to various portions of conversation 602. Additionally, user interface 600 may include a plurality of control elements 612 that enable interaction with conversation 602. For example, if the operator is unsatisfied with response 608, the operator may select for the chatbot to “Retry”. In some examples, this selection may be received and stored as feedback data 418 that is utilized to further fine-tune or otherwise retrain LLM 234. Other example control elements 612 can include an “Undo” graphical element to remove a latest request 606 or response 608 displayed within conversation 602 or a “Clear” graphical element to remove an entirety of conversation 602 from display.

User interface 600 described above is provided merely as an example, and may include additional, fewer, different, or differently arranged information and/or interactive control elements than depicted in FIG. 6.

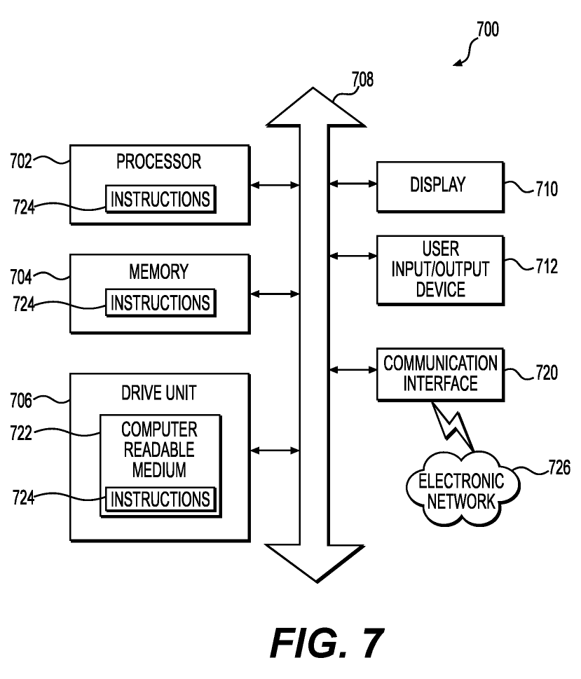

FIG. 7 depicts an example of a computer 700. FIG. 7 is a simplified functional block diagram of computer 700 that may be configured as a device for executing processes, steps, or operations depicted in, or described with respect to, FIGS. 2A-6 and, according to exemplary aspects described herein. For example, computer 700 may be configured as one or more of medical device 102, computing system 104, headset 105, display device(s) 106, edge device(s) 130, server-side system(s) 140, and/or another device or component according to exemplary aspects described herein. In various aspects, any of the systems herein may be or include computer 700 including, e.g., a data communication interface 720 for packet data communication. Computer 700 may communicate with one or more other computers, for example, using an electronic network 726 (e.g., via data communication interface 720). Electronic network 726 may include a wired or wireless network, for example, similar to network 150 depicted in FIG. 1.

Computer 700 also may include a central processing unit ("CPU"), in the form of one or more processors 702, for executing program instructions 724. In some examples, processors 702 may be or include one or more field-programmable gate arrays (FPGAs). Program instructions 724 may include at least instructions for request processing.

Computer 700 may include an internal communication bus 708. Computer 700 may also include a drive unit 706 (such as read-only memory (ROM), hard disk drive (HDD), solid-state disk drive (SDD), etc.) that may store data on a computer readable medium 722 (e.g., a non-transitory computer readable medium), although computer 700 may receive programming and data via network communications. Computer 700 may also have a memory 704 (such as random-access memory (RAM)) storing instructions 724 for executing techniques presented herein. It is noted, however, that in some aspects, instructions 724 may be stored temporarily or permanently within other modules of computer 700 (e.g., processor 702 and/or computer readable medium 722). Computer 700 also may include user input and output devices 712 and/or a display 710 to connect with input and/or output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as "products" or "articles of manufacture" typically in the form of executable code and/or associated data that is carried on or embodied in a type of machine-readable medium. "Storage" type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible "storage" media, terms such as computer or machine "readable medium" refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is Claimed is:

1. A method for voice request processing, the method comprising:
receiving a recording including a voice request captured by a medical system during a medical procedure;
transcribing audio content of the recording to text;
generating a response to the voice request by:
determining, based on the text, to invoke a tool from a plurality of tools, the tool having a function associated with the voice request;
responsive to a call to the tool, receiving a result generated from an execution of the function by the tool based on inputs provided as part of the call, the inputs including at least a portion of the text; and
generating the response to the voice request based on the text and the result; and
providing the response to the medical system for presentation on a display of the medical system.

2. The method of claim 1, wherein the response is generated using a large language model (LLM), and generating the response to the voice request comprises:
in response to receiving the text as part of a prompt, generating and including, by the LLM, code in an initial output of the LLM, the code indicating the tool to be invoked;
calling, by an agent, the tool based on the code included in the initial output of the LLM;
in response to receiving the result, augmenting, by the agent, the prompt to include the result to generate an augmented prompt for provision to the LLM; and
processing, by the LLM, the augmented prompt to generate the response to the voice request.

3. The method of claim 1, wherein the response is generated using a large language model (LLM) fine-tuned to determine the tool, from the plurality of tools, to be invoked based on one or more tokens identified in the text that are associated with the tool.

4. The method of claim 1, wherein the voice request is to control an operating parameter of a component of the medical system, the function of the tool is associated with generating a control command for the operating parameter, and generating the response comprises:
receiving the result, including the control command having a syntax specific to the medical system; and
including the control command in the response, wherein, when the response is provided to the medical system, the medical system is configured to read and execute the control command based on the syntax.

5. The method of claim 4, wherein:
the medical system includes an endoscope having an imaging device and a lighting device and a computing system that the endoscope is removably connected to;
the operating parameter is an operating parameter of one of the imaging device or the lighting device; and
the syntax of the control command is specific to the computing system such that, upon receiving the response, the computing system is configured to generate and transmit one or more control signals to the one of the imaging device or the lighting device to control the operating parameter.

6. The method of claim 1, wherein the voice request is a request to retrieve a video associated with an aspect of the medical procedure for playback on a display component of the medical system during the medical procedure, and the function of the tool is associated with interacting with a video retrieval system, the video retrieval system configured to:
transform the text to generate transformed text in a format compatible with a video search data store, the video search data store configured to store, for each video of a plurality of videos, one or more transformed portions of video transcript corresponding to one or more portions of video data of the video;
identify, from the video search data store and based on the transformed text, a transformed portion of video transcript relevant to the transformed text, the transformed portion of video transcript being associated with the aspect of the medical procedure; and
generate, as the result, a portion of video transcript corresponding to the transformed portion of video transcript, a timestamp associated with the portion of video transcript, and video data associated with the portion of video transcript, wherein the response includes at least the timestamp and the video data and, upon receiving the response, the medical system plays back the video data at a time corresponding to the timestamp.

7. The method of claim 6, wherein the response further includes the portion of video transcript, and the medical system displays the portion of video transcript in addition to the playback of the video data at the time corresponding to the timestamp.

8. The method of claim 6, wherein the video retrieval system is further configured to establish the video search data store by, for each video of the plurality of videos:
receiving video data of the video;
generating a video transcript from the video data;
extracting metadata associated with the video data, including a plurality of timestamps;
storing the video data, the video transcript, and the metadata in a video data store, wherein the plurality of timestamps is stored in association with a corresponding plurality of portions of the video transcript;
transforming one or more of the plurality of portions of the video transcript to generate the one or more transformed portions of the video transcript; and
storing the one or more transformed portions of the video transcript in the video search data store.

9. The method of claim 6, wherein the video retrieval system is further configured to:
identify, from a medical search data store and based on the transformed text, a transformed portion of medical data relevant to the transformed text, the transformed portion of medical data being associated with the aspect of the medical procedure;
determining a relevancy of each of the transformed portion of video transcript and the transformed portion of medical data to the request; and
based on the relevancy, determining to include at least the portion of video transcript corresponding to the transformed portion of video transcript in the result.

10. The method of claim 9, wherein, based on the relevancy, determining to further include the transformed portion of medical data in the result.

11. The method of claim 1, wherein the response is generated using a large language model (LLM) implementing retrieval augmentation generation (RAG), and generating the response to the voice request comprises:
obtaining, from one or more contextual data stores, additional information for use in generating the response.

12. The method of claim 11, wherein one of the one or more contextual data stores is a direction for use (DFU) data store configured to store one or more documents associated with use of the medical system, the voice request is a query related to use of a component of the medical system, and generating the response to the voice request comprises:
identifying, from the one or more documents within the DFU data store, information associated with the use of the component of the medical system; and
including at least a portion of the information in the response.

13. The method of claim 1, wherein the recording including the voice request is a first recording, the voice request is to record and transcribe the medical procedure, the response includes a command that is read and executed by the medical system to cause the medical system to generate a second recording of the medical procedure, and the method further comprises:
receiving the second recording;
transcribing audio content of the second recording to text to generate a medical procedure transcript; and
automatically storing the medical procedure transcript in association with an electronic record of a patient.

14. The method of claim 1, wherein the medical system includes a microphone, and the voice request is captured and converted, by the microphone, to electrical signals comprising the recording.

15. The method of claim 14, wherein the medical system includes a computing system, and the microphone is one of integrated with the computing system or a headset communicatively coupled to the computing system.

16. A method for request processing, the method comprising:
receiving a request, captured by a medical system, to control an operating parameter of a component of the medical system during a medical procedure;
generating a response to the request by:
determining, based on a text of the request, to invoke a tool from a plurality of tools, the tool having a function associated with generating a control command for the operating parameter;
responsive to a call to the tool, receiving a result generated from an execution of the function by the tool, the result including the control command having a syntax specific to the medical system; and
generating the response to the request based on the text and the result, the response including the control command; and
providing the response to the medical system, wherein the medical system reads and executes the control command based on the syntax.

17. The method of claim 16, wherein the request is a voice request, and the method further comprises:
receiving a recording including the voice request captured by the medical system; and
transcribing audio content of the recording to the text for use in generating the response.

18. The method of claim 16, wherein:
the medical system includes an endoscope having an imaging device and a lighting device and a computing system that the endoscope is removably connected to;
the operating parameter is an operating parameter of one of the imaging device or the lighting device; and
the syntax of the control command is specific to the computing system such that, upon receiving the response, the computing system is configured to generate and transmit one or more control signals to the one of the imaging device or the lighting device to control the operating parameter.

19. A method for request processing, the method comprising:
receiving a request, the request captured by a medical system, to retrieve a video associated with an aspect of a particular type of medical procedure for playback on a display component of the medical system during a medical procedure of the particular type;
transforming text of the request to generate transformed text in a format compatible with a video search data store, the video search data store configured to store, for each video of a plurality of videos, one or more transformed portions of video transcript corresponding to one or more portions of video data of the video;
identifying, from the video search data store and based on the transformed text, a transformed portion of video transcript relevant to the transformed text, the transformed portion of video transcript being associated with the aspect of the particular type of medical procedure;
generating a response to the request that includes a portion of video transcript corresponding to the transformed portion of video transcript, a timestamp associated with the portion of video transcript, and video data associated with the portion of video transcript; and
providing the response to the medical system, wherein the medical system plays back the video data at a time corresponding to the timestamp.

20. The method of claim 19, further comprising:
establishing the video search data store by, for each video of the plurality of videos:
receiving video data of the video;
generating a video transcript from the video data;
extracting metadata associated with the video data, including a plurality of timestamps;
storing the video data, the video transcript, and the metadata in a video data store, wherein the plurality of timestamps is stored in association with a corresponding plurality of portions of the video transcript;
transforming one or more of the plurality of portions of the video transcript to generate the one or more transformed portions of the video transcript; and
storing the one or more transformed portions of the video transcript in the video search data store.
